SEQUENCE LISTING (3 sequences)

SEQ ID NO: 1 - RNA, 19 nt, Homo sapiens
cccaucuugaacauaccag

SEQ ID NO: 2 - RNA, 20 nt, Homo sapiens
aaccggugcacagagggugu

SEQ ID NO: 3 - RNA, 20 nt, Homo sapiens
auuuacaagcuguacauggg

(19) United States
(12) Patent Application Publication
(10) Pub. No.: US 2023/0210903 A1
(43) Pub. Date: Jul. 6, 2023

(54) HYPOXIA-RESISTANT NATURAL KILLER CELLS

(71) Applicant: ONK THERAPEUTICS LIMITED, Galway (IE)

(72) Inventors: Michael Eamon Peter O'DWYER, Galway (IE); Jinsong HU, Xi'an, Shaanxi (CN)

(21) Appl. No.: 18/000,637

(22) PCT Filed: Jun. 2, 2020
(86) PCT No.: PCT/EP2020/065228

§ 371 Date: Dec. 2, 2022

(51) Int. Cl. A61K 35/17; C12N 15/90; C12N 5/0783; A61P 35/00; C12N 9/16

(52) U.S. Cl. CPC A61K 35/17; C12N 15/90; C12N 5/0646; A61P 35/00; C12Y 301/03048; C12N 9/16; C12N 2510/00

(57) ABSTRACT

NK cells and NK cell lines are modified so as to have a more cytotoxic phenotype, namely to have reduced function of Src homology 2 domain-containing protein tyrosine phosphatase 1 (SHP-1). Methods of making the modified cells and cell lines, compositions comprising the modified cells and cell lines, as well as uses of said cells, cell lines and compositions in therapy are also provided.

### INTRODUCTION

The present invention relates to the modification of natural killer (NK) cells and NK cell lines to produce derivatives thereof with a more cytotoxic phenotype. Furthermore, the present invention relates to methods of producing modified NK cells and NK cell lines, compositions containing the cells and cell lines and uses of said compositions in therapy.

### BACKGROUND TO THE INVENTION

NK cell cytotoxicity refers to the ability of NK cells to induce target cell death, e.g. by releasing cytolytic compounds or by binding receptors on cancer cell membranes and inducing apoptosis of said target cells. Cytotoxicity is affected not only by signals that induce release of cytolytic compounds but also by signals that inhibit their release. An increase in cytotoxicity will therefore lead to more efficient killing of target cells, with a lower chance of the target cell (particularly when a cancer cell) dampening the cytotoxic activity of the NK cell.

It is known that intracellular signals activating NK cell cytotoxicity are propagated primarily through protein phosphorylation of extracellular signal-regulated kinase (ERK) and signal transducer and activator of transcription 3 (STAT3) (Cacalano. “Regulation of Natural Killer Cell Function by Stat3” Front Immunol. Vol. 7, pp. 128, 2016; and Jiang, et al. “Pivotal Role of Phosphoinositide-3 Kinase in Regulation of Cytotoxicity in Natural Killer Cells” Nat Immunol. Vol. 1, no. 5, pp. 419-25, 2000).

A considerable amount of research into NK cell cytotoxicity has been performed using mouse models. One example is the finding that perforin and granzyme B mRNA are constitutively transcribed in mouse NK cells, but minimal levels of protein are detected until stimulation or activation of the NK cells (Fehniger et al, 2007). Although this work and other work using mouse NK cells is of interest, it cannot be relied upon as conclusive evidence for NK cell cytotoxicity in humans. In contrast to the above example, human NK cells express high levels of perforin and granzyme B protein prior to stimulation (Leong et al, 2011). The result being that when either mouse or human NK cells are freshly isolated in culture, the mouse NK cells have weak cytolytic activity, whereas the human NK cells exhibit strong cytolytic capabilities.

Mouse and human NK cells also vary greatly in their expression markers, signalling cascades and tissue distribution. For example, CD56 is used as a marker for human NK cells, whereas mouse NK cells do not express this marker at all. Furthermore, a well-established mechanism for regulating NK cell cytotoxicity is via ligand binding NK activation and inhibitory receptors. Two of the most prominent human NK activation receptors are known to be NKp30 and NKp44, neither of which are expressed on mouse NK cells. With regards to NK inhibitory receptors, whilst human NK cells express KIRs that recognise MHC class I and dampen cytotoxic activity, mouse NK cells do not express KIRs at all but, instead, express Ly49s (Trowsdale et al, 2001). All in all, despite mouse NK cells achieving the same function as human NK cells in their natural physiological environment, the mechanisms that fulfil this role vary significantly between species.

Cancer cells have a unique ability to evade immune responses, for example via expressing ligands for inhibitory receptors and by expressing TRAIL decoy receptors. Cancer cells can also show resistance to therapies based on targeted chimeric antigen receptors (CARs) by reducing expression of the target. Further still, many cancers form tumours with a restricted blood supply, establishing a hypoxic environment in which immune effector cells struggle to perform their anti-cancer roles.

Thus, there exists a need for alternative and preferably improved human NK cells and human NK cell lines, e.g. with a more cytotoxic profile and a reduced susceptibility to evasion by cancer cells.

An object of the invention is to provide NK cells and NK cell lines with a more cytotoxic phenotype. A further object is to provide methods for producing modified NK cells and NK cell lines, compositions containing the cells or cell lines and uses of said cells and compositions in therapy. More particular embodiments aim to provide treatments for identified cancers, e.g. blood cancers and solid cancers. Specific embodiments aim at combining multiple modifications of NK cells and NK cell lines to further enhance the cytotoxicity of the modified cells and/or reduce the extent to which cancers can evade NK cell-based therapies.

### SUMMARY OF THE INVENTION

The invention provides modified NK cells and NK cell lines with a more cytotoxic phenotype (particularly in hypoxic environments), methods of making the modified cells and cell lines, compositions comprising the modified cells and cell lines, as well as uses of said cells, cell lines and compositions in therapy.

The invention provides NK cells and NK cell lines modified to have reduced function of Src homology 2 domain-containing protein tyrosine phosphatase 1 (SHP-1). Further modifications may also be present, providing the NK cells and NK cell lines with a more cytotoxic phenotype.

The invention further provides methods of treating cancer, e.g. blood cancer, using the modified NK cells and NK cell lines.

Diseases particularly treatable according to the invention include cancers, e.g. solid cancers. Tumours and cancers in humans in particular can be treated. References to tumours herein include references to neoplasms.

### DETAILS OF THE INVENTION

Accordingly, the present invention provides an NK cell that has been modified so as to increase its cytotoxicity (particularly in hypoxic environments).

References to NK cells herein include autologous NK cells, umbilical cord-derived NK cells, allogeneic NK cells, iPSC-derived NK cells, and NK cell lines such as KHYG-1 and NK-92. Preferably, the NK cells are human NK cells.

Increased or enhanced cytotoxicity resulting from modification of an NK cell is defined by comparison to the cytotoxicity of a wildtype NK cell not having such a modification. A wildtype cell is defined as a cell of the same type as that comprising the modification but not having the modification itself.

Preferably, the NK cell of the invention is modified to have reduced function of Src homology 2 domain-containing protein tyrosine phosphatase 1 (SHP-1). The modification is preferably a genetic modification.

Optionally, the modification involves knocking out SHP-1 expression. Preferably, however, SHP-1 expression is knocked down (as opposed to being knocked out entirely). SHP-1 expression is preferably knocked down by at least 50%, more preferably at least 60%, more preferably at least 70%, more preferably at least 80%, more preferably at least 90%, compared to the expression of SHP-1 in wildtype NK cells. As such, it is preferred that some residual SHP-1 expression remains following modification of the NK cell.

It is preferred that the modification reducing SHP-1 function is a transient modification, e.g. the modification is temporary and/or reversible. This is particularly advantageous when modifying immature NK cells which may rely on SHP-1 expression for development.

Preferably, the transient modification is achieved using RNA interference (RNAi), e.g. using siRNA or shRNA.

Optionally, SHP-1 function is reduced by expressing an inactive or partially active form of SHP-1. This modified form competes with wildtype SHP-1 but without performing the same level of tyrosine phosphorylation associated with the wildtype. Preferably, the inactive form is expressed from an extra-chromosomal nucleic acid. More preferably, the extra-chromosomal nucleic acid is RNA. It is preferred that the inactive form of SHP-1 is expressed via an mRNA-based transfection system, e.g. the Maxcyte GT system though others are also suitable.

It is thus preferred that SHP-1 function is reduced by expressing a dominant negative form of SHP-1. Preferably the dominant negative form is inducible, e.g. by doxycycline or an antibody. Genetic switches may be used to turn expression on and off, or even tune expression levels.

Expression of genetic material in NK cells, e.g. shRNAs and/or dominant negative forms of SHP-1, is preferably drug-inducible. Preferably, induction is via a genetic circuit platform that acts as a one-time state switch in NK cells. Preferably, the circuit has memory capability that reduces the need for prolonged drug administration to maintain gene expression levels. Preferably, a variety of circuits, including an ON switch, an OFF switch and an Expression Level switch are provided, meaning gene activity can be tuned via drug dosage and drug exposure duration.

SHP-1 function may also be reduced using a phosphatase inhibitor. Preferably, the inhibitor is specific for tyrosine phosphatases. The phosphatase inhibitor is preferably reversible. Examples of suitable phosphatase inhibitors include tyrosine phosphatase inhibitor 1 (TPI-1), sodium stibogluconate (SSG), sodium orthovanadate (SOV) and sodium fluoride (NaF).

The NK cell of the invention may further be modified to express TRAIL ligand (in addition to any TRAIL ligand naturally expressed by the NK cell) and/or to express a chimeric antigen receptor (CAR).

A CAR comprises a leader sequence, a targeting domain, a transmembrane domain, and one or more intracellular domains. The leader sequence is cleaved in the endoplasmic reticulum and thus does not form part of the mature CAR as expressed on the cell surface. The targeting domain of the CAR is often derived from a single chain variable fragment (scFv) of an antibody. As is well known in the art, a scFv comprises the variable heavy chain (VH) of an antibody linked to the variable light chain (VL) of an antibody. The transmembrane (TM) domain of the CAR functions to anchor the CAR to the cell membrane. The intracellular domain of the CAR optionally works to increase potency through immune cell signal transduction. As such, a common intracellular domain used in the construction of CARs is that from CD3zeta of the T cell receptor. Furthermore, it is known to include more than one intracellular domain in the CAR, in order to provide a highly potent CAR. Co-stimulatory domains (e.g. 4-1BB and CD28) are excellent choices for this purpose.

Suitably, the CAR binds an antigen expressed on one or more cancer cells, e.g. CD38, CD319/SLAMF-7, TNFRSF17/BCMA, SYND1/CD138, CD229, CD47, Her2/Neu, epidermal growth factor receptor (EGFR), CD123/IL3-RA, CD19, CD20, CD22, Mesothelin, EpCAM, MUC1 (including aberrantly glycosylated forms thereof), MUC16, Tn antigen, NEU5GC, NeuGcGM3, GD2, CLL-1, or HERV-K. When referring to the CAR herein, reference to the targeted antigen indicates CAR function. Thus, a “CD19 CAR” is a CAR that binds to CD19 expressed on the surface of a target cell, usually a cancer cell.

Preferably, the CAR binds CD19, MUC-1 or CD38.

The NK cell of the invention may express wildtype TRAIL ligand, in which case it preferably overexpresses wildtype TRAIL ligand. Preferably, expression of the wildtype TRAIL ligand is increased at least 1.5-fold, more preferably at least 2-fold, more preferably at least 5-fold, more preferably at least 10-fold, compared to expression of wildtype TRAIL on the wildtype NK cell.

The NK cell of the invention may express mutant TRAIL ligand, also referred to as variant TRAIL ligand or just TRAIL variant; in these cases, it is preferred that the NK cell expresses a TRAIL mutant/variant with increased affinity for TRAIL death receptors, e.g. DR4 and/or DR5, compared with the affinity of wildtype TRAIL ligand for TRAIL death receptors. The mutants/variants also preferably have lower affinity (or in effect no affinity) for ‘decoy’ receptors, compared with the binding of wildtype TRAIL to decoy receptors. Such decoy receptors represent a class of TRAIL receptors that bind TRAIL ligand but do not have the capacity to initiate cell death and, in some cases, act to antagonize the death signaling pathway.

Wildtype TRAIL is typically known to have a KD of >2 nM for DR4, >5 nM for DR5 and >20 nM for the decoy receptor DcR1 (WO 2009/077857; measured by surface plasmon resonance), or around 50 to 100 nM for DR4, 1 to 10 nM for DR5 and 175 to 225 nM for DcR1 (Truneh, A. et al. 2000; measured by isothermal titration calorimetry and ELISA). Therefore, an increased affinity for DR4 is suitably defined as a KD of <2 nM or <50 nM, respectively, whereas an increased affinity for DR5 is suitably defined as a KD of <5 nM or <1 nM, respectively. A reduced affinity for decoy receptor DcR1 is suitably defined as a KD of >50 nM or >225 nM, respectively. In any case, an increase or decrease in affinity exhibited by the TRAIL variant/mutant is relative to a baseline affinity exhibited by wildtype TRAIL. The affinity is preferably increased at least 10%, more preferably at least 25%, more preferably at least 50%, more preferably at least 100%, more preferably at least 500%, compared with that exhibited by wildtype TRAIL.

In certain embodiments, the TRAIL variant comprises at least one amino acid substitution at a position selected from the group consisting of 131, 149, 159, 160, 189, 191, 193, 195, 199, 200, 201, 203, 204, 212, 213, 214, 215, 218, 240, 251, 261, 264, 266, 267, 269, and 270.

In certain embodiments, the TRAIL variant comprises at least one substitution selected from the group consisting of G131R, G131K, R149I, R149M, R149N, R149K, S159R, G160E, Y189A, Y189Q, R191K, Q193H, Q193K, Q193S, Q193R, E195R, N199V, N199R, N199H, T200H, K201R, K201H, D203A, K204E, K204D, K204Y, K212R, Y213W, T214R, S215D, S215E, S215H, S215K, S215D, D218H, D218A, Y240A, K251D, K251E, K251Q, T261L, H264R, 1266L, D267Q, D269A, D269H, and H270D.

In certain embodiments, the TRAIL variant comprises at least two substitutions selected from the group consisting of G131R, G131K, R149I, R149M, R149N, R149K, S159R, G160E, Y189A, Y189Q, R191K, Q193H, Q193K, Q193S, Q193R, E195R, N199V, N199R, N199H, T200H, K201R, K201H, D203A, K204E, K204D, K204Y, K212R, Y213W, T214R, S215D, S215E, S215H, S215K, S215D, D218H, D218A, Y240A, K251D, K251E, K251Q, T261L, H264R, 1266L, D267Q, D269A, D269H, and H270D.

In certain embodiments, the TRAIL variant comprises at least three substitutions selected from the group consisting of G131R, G131K, R149I, R149M, R149N, R149K, S159R, G160E, Y189A, Y189Q, R191K, Q193H, Q193K, Q193S, Q193R, E195R, N199V, N199R, N199H, T200H, K201R, K201H, D203A, K204E, K204D, K204Y, K212R, Y213W, T214R, S215D, S215E, S215H, S215K, S215D, D218H, D218A, Y240A, K251D, K251E, K251Q, T261L, H264R, 1266L, D267Q, D269A, D269H, and H270D.

In certain embodiments, amino acid substitution of the TRAIL variant is selected from the group consisting of G131R, G131K, R149I, R149M, R149N, R149K, S159R, G160E, Y189A, Y189Q, R191K, Q193H, Q193K, Q193S, Q193R, E195R, N199V, N199R, N199H, T200H, K201R, K201H, D203A, K204E, K204D, K204Y, K212R, Y213W, T214R, S215D, S215E, S215H, S215K, S215D, D218H, D218A, Y240A, K251D, K251E, K251Q, T261L, H264R, I266L, D267Q, D269A, D269H, H270D, T214R/E195R, T214R/D269H, Y189A/Q193S/N199V/K201R/Y213W/S215D, Y213W/S215D, N199R/K201H, N199H/K201R, G131R/N199R/K201H, G131R/N199R/K201H/R149I/S159R/S215D, G131R/R149I/S159R/S215D, G131R/N199R/K201H/R149I/S159R/S215D, G131R/D218H, Y189Q/R191K/Q193R/H264R/I266L/D267Q, T261L/G160E, T261L/H270D, T261L/G160E/H270D, and T261L/G160E/H270D/T200H (use of “/” indicates multiple amino acid substitutions).

In certain embodiments, amino acid substitution of the TRAIL variant is selected based on the variant having an increased affinity for DR5, a substitution of this kind may be selected from the group consisting of D269H, E195R, T214R, D269H/E195R, T214R/E195R, T214R/D269H, N199V, Y189A/Q193S/N199V/K201R/Y213W/S215D, Y213W/S215D, D269A and Y240A.

In certain embodiments, amino acid substitution of the TRAIL variant is selected based on the variant having an increased affinity for DR4; a substitution of this kind may be selected from the group consisting of G131R, G131K, R149I, R149M, R149N, R149K, S159R, Q193H, W193K, N199R, N199R/K201H, N199H/K201R, G131R/N199R/K201H, G131R/N199R/K201H, G131R/N199R/K201H/R149I/S159R/S215D, G131R/R149I/S159R/S215D, G131R/D218H, K201R, K201H, K204E, K204D, K204L, K204Y, K212R, S215E, S215H, S215K, S215D, D218H, K251 D, K251E, K251Q and Y189Q/R191K/Q193R/H264R/I266L/D267Q.

In certain embodiments, amino acid substitution of the TRAIL variant is selected based on the variant having a decreased affinity for TRAIL decoy receptors; a substitution of this kind may be selected from the group consisting of T261L, H270D, T200H, T261 L/G160E, T261L/H270D, T261L/G160E/H270D, T261L/G160E/H270D/T200H, D203A and D218A.

In an optional embodiment, treatment of a cancer using modified NK cells expressing TRAIL and/or TRAIL variant is enhanced by administering to a patient an agent capable of upregulating expression of TRAIL death receptors on cancer cells. This agent may be administered prior to, in combination with or subsequently to administration of the modified NK cells. It is preferable, however, that the agent is administered prior to administering the modified NK cells. In a preferred embodiment the agent upregulates expression of DR5 and/or DR4 on cancer cells. The agent may optionally be a chemotherapeutic medication, e.g. Bortezomib, and administered in a low dose capable of upregulating TRAIL receptor expression on the cancer. The invention is not limited to any particular agents capable of upregulating TRAIL receptor expression, but examples of agents include SMAC mimetics, Bortezomib, Gefitinib, Piperlongumine, Doxorubicin, Alpha-tocopheryl succinate and HDAC inhibitors.

According to a preferred embodiment of the invention, the TRAIL ligand/mutant TRAIL ligand is linked to one or more NK cell co-stimulatory domains, e.g. 4-1BB, CD28, 2B4, DAP-10, DAP-12, CD278 (ICOS) and/or OX40. Binding of the ligand to its receptor on a target cell thus promotes apoptotic signals within the target cell, as well as stimulating cytotoxic signals in the NK cell.

It is also preferred that the intracellular domain of the CAR comprises one or more co-stimulatory domains, e.g. CD3zeta, 4-1BB, CD28, 2B4, DAP-10, DAP-12, CD278 (ICOS) and/or OX40.

The NK cells of the invention may also be modified to have increased resistance to TRAIL-induced cell death. The cells may be less vulnerable to TRAIL-induced cell death or fratricide as a result.

The NK cells may be modified to have reduced function of one or more TRAIL receptors. This is optionally achieved using gene knockout or knockdown (e.g. using siRNA) or restricting the expression of the TRAIL receptor within the cell endoplasmic reticulum. Preferably, DR4 and/or DR5 function is reduced on the NK cells of the invention. It is particularly preferred that the DR4 and/or DR5 genes are knocked out. If multiple copies of the genes are present, it is preferred that all are knocked out.

The NK cells may be modified in a way that both reduces TRAIL-induced death of the cells and provides the cells with a more cytotoxic phenotype. Preferably the same modification can achieve both of these advantages. It is preferred that the NK cells are modified to express a TRAIL receptor linked to a co-stimulatory domain. The cells may express a TRAIL receptor linked to one or more co-stimulatory domains. Preferably, the TRAIL receptor is selected from DR4 and DR5. Preferably, the co-stimulatory domain is selected from one or more of 4-1BB, CD28, 2B4, DAP-10, DAP-12, CD278 (ICOS) and OX40. More preferably, the co-stimulatory domain is 4-1BB linked to CD3zeta.

The resistance of the modified NK cells against TRAIL-induced cell death is preferably increased by at least 5%, more preferably at least 10%, more preferably at least 25%, most preferably at least 50%, relative to wildtype NK cells. Resistance to cell death can be measured in a number of ways known to the skilled person, e.g. by performing a cell viability assay. Preferably, increased resistance to cell death is measured using a flow cytometric propidium iodide cell viability assay. In an example, an NK cell population modified according to the invention to exhibit at least 10% increased resistance to TRAIL-induced cell death would be identified through an assay where soluble TRAIL is incubated with (1) the modified cells and (2) the wildtype cells, and then after staining each cell population with propidium iodide, the modified cell population is found to have a cell viability at least 10% higher than the wildtype population.

Optionally, the NK cells are further modified to have reduced or absent checkpoint inhibitory receptor function. Preferably, these receptors are specific checkpoint inhibitory receptors. Preferably still, these checkpoint inhibitory receptors are one or more or all of CD96 (TACTILE), CD152 (CTLA4), CD223 (LAG-3), CD279 (PD-1), CD328 (SIGLEC7), SIGLEC9, and/or TIGIT. Reduced or absent checkpoint inhibitory receptor function may be achieved, for example, by knocking down or knocking out one or more checkpoint inhibitory receptor genes.

The NK cells are optionally further modified to express an Fc receptor, in addition to any Fc receptor naturally expressed by the NK cells. Preferably, the Fc receptor is CD16. More preferably, the Fc receptor is a high-affinity variant of CD16, e.g. CD16 having a valine at amino acid position 158.

The cells may be modified by genetic modification. Optionally, this modification occurs before the cell has differentiated into an NK cell. For example, pluripotent stem cells (e.g. iPSCs) can be genetically modified to express a TRAIL variant before being differentiated into NK cells.

As per the objects of the invention, the modified NK cell, modified NK cell line, or composition thereof is for use in therapy. Preferably, the cells are used for treating cancer in a patient, especially blood cancers and solid cancers. Alternatively, the NK cells may be used to treat a viral infection, e.g. HIV or COVID-19.

NK cells of the present invention are suitably used to treat a disease or condition associated with hypoxia, e.g. a tumour in a hypoxic environment. It has been shown by the present inventors that hypoxic environments are capable of diminishing NK cell cytotoxicity. Advantageously, the NK cells of the invention with reduced SHP-1 function have been shown to have increased cytotoxicity against cancer cells in hypoxic environments. In other words, NK cells of the invention demonstrate increased resistance to the negative effects of hypoxia on NK cell cytotoxicity.

In preferred embodiments, the modified NK cell, NK cell line or composition is for use in treating solid cancers including cancers of the breast, brain, colon, head and/or neck, liver, lung, pancreas, prostate and kidney.

In preferred embodiments, the modified NK cell, NK cell line or composition is for use in treating blood cancers including acute lymphocytic leukemia (ALL), acute myeloid leukemia (AML), chronic lymphocytic leukemia (CLL), chronic myeloid leukemia (CML), Hodgkin's lymphoma, non-Hodgkin's lymphoma, including T-cell lymphomas and B-cell lymphomas, multiple myeloma, asymptomatic myeloma, smoldering multiple myeloma (SMM), active myeloma and light chain myeloma. Preferably, the cancer is a human cancer.

Various routes of administering the modified NK cells to a patient in need thereof will be known to the skilled person. Administration of the modified NK cells can be systemic or localized, e.g. via the intraperitoneally or intratumourally (suitable especially for solid tumours).

Advantageously, an NK cell, preferably treated to reduce its tumourigenicity, for example by rendering it mortal and/or incapable of dividing, can be obtained from a blood cancer cell line and used in methods of the invention to treat cancer.

To render a cancer-derived cell more acceptable for therapeutic use, it is generally treated or pre-treated in some way to reduce or remove its propensity to form tumours in the patient. Specific modified NK cell lines used in examples are safe because they have been rendered incapable of division; they are irradiated and retain their killing ability but die within about 3-4 days. Specific cells and cell lines are hence incapable of proliferation, e.g. as a result of irradiation. Treatments of potential NK cells for use in the methods herein include irradiation to prevent them from dividing and forming a tumour in vivo and genetic modification to reduce tumourigenicity, e.g. to insert a sequence encoding a suicide gene that can be activated to prevent the cells from dividing and forming a tumour in vivo. Suicide genes can be turned on by exogenous, e.g. circulating, agents that then cause cell death in those cells expressing the gene. A further alternative is the use of monoclonal antibodies targeting specific NK cells of the therapy. CD52, for example, is expressed on KHYG-1 cells and binding of monoclonal antibodies to this marker can result in antibody-dependent cell-mediated cytotoxicity (ADCC) and KHYG-1 cell death.

As discussed in an article published by Suck et al, 2006, cancer-derived NK cells and cell lines are easily irradiated using irradiators such as the Gammacell 3000 Elan. A source of Cesium-137 is used to control the dosing of radiation and a dose-response curve between, for example, 1 Gy and 50 Gy can be used to determine the optimal dose for eliminating the proliferative capacity of the cells, whilst maintaining the benefits of increased cytotoxicity. This is achieved by assaying the cells for cytotoxicity after each dose of radiation has been administered.

There are significant benefits of using an irradiated NK cell line for adoptive cellular immunotherapy over the well-established autologous or MHC-matched T cell approach. Firstly, the use of a NK cell line with a highly proliferative nature means expansion of modified NK cell lines can be achieved more easily and on a commercial level. Irradiation of the modified NK cell line can then be carried out prior to administration of the cells to the patient. These irradiated cells, which retain their useful cytotoxicity, have a limited life span and, unlike modified T cells, will not circulate for long periods of time causing persistent side-effects.

Additionally, the use of allogeneic modified NK cells and NK cell lines means that MHC class I expressing cells in the patient are unable to inhibit NK cytotoxic responses in the same way as they can to autologous NK cytotoxic responses. The use of allogeneic NK cells and cell lines for cancer cell killing benefits from the GVL effect and, unlike for T cells, allogeneic NK cells and cell lines do not stimulate the onset of GVHD, making them a much preferred option for the treatment of cancer via adoptive cellular immunotherapy.

### EXAMPLES

The present invention is now described in more and specific details in relation to the production of NK cells, modified to exhibit increased cytotoxic activity, wherein specific embodiments are illustrated with reference to the accompanying drawings in which:

FIG. 1a-1d show the effect of hypoxia on NK cell cytotoxicity;

FIG. 2a-1d show the effect of hypoxia on the expression of various cytotoxicity-related markers in NK cells;

FIG. 3a-3e show the effect of hypoxia on the phosphorylation levels of ERK and STAT3 in NK cells;

FIG. 4a-4e show the effect of hypoxia on SHP-1 and SHP-2 activation in NK cells;

FIG. 5a-5c show the effect of inhibiting SHP-2 on ERK signalling, STAT3 signalling and NK cell cytotoxicity;

FIG. 6a-6c show the effect of inhibiting SHP-1 on ERK signalling, STAT3 signalling and NK cell cytotoxicity;

FIG. 7 shows the baseline expression of TRAIL on KHYG-1 cells;

FIG. 8 shows the expression of TRAIL and TRAIL variant after transfection of KHYG-1 cells;

FIG. 9 shows the expression of CD107a after transfection of KHYG-1 cells;

FIG. 10 shows the effects of transfecting KHYG-1 cells with TRAIL and TRAIL variant on cell viability;

FIG. 11 shows the baseline expression of DR4, DR5, DcR1 and DcR2 on both KHYG-1 cells and NK-92 cells;

FIGS. 12-14 show the effects of expressing TRAIL or TRAIL variant in KHYG-1 cells on apoptosis of three target cell populations: K562, RPMI8226 and MM1.S, respectively; and

FIG. 15 shows mitigation of NK cell fratricide by knocking down DR5 expression.

DNA, RNA and amino acid sequences are referred to below, in which:
SEQ ID NO: 1 is an example gRNA for DR5;
SEQ ID NO: 2 is an example gRNA for DR4; and
SEQ ID NO: 3 is a second example gRNA for DR4.

### Examples 1-3: Detailed Figure Overview

FIG. 1

Hypoxic NK cells show lower cytotoxicity against tumour cells. (a-b) Flow cytometric analysis of KHYG-1 cells cytotoxicity against tumour cells. KHYG-1 cells were incubated with K562 (a) or MM.1S (b) tumour cells for 4 h at different E:T ratios after cultivation at normoxic (20% 02) and hypoxic (1% 02) conditions for 24 h. Left panel: a representation of results from three experiments; Right panel: statistical analysis showing the percentage of tumour cells killed by NK cells (n=3, * P<0.05). (c) Western blotting analysis of the effects of hypoxia on the expression of hypoxia marker HIF-1α. NK cells were cultured in 20% or 1% 02 for 24 h, and then western blotting analysis was performed. (d) Flow cytometric analysis of the effects of hypoxia on NK cell viability by performing Annexin V-FITC/7-AAD staining. NK cells were cultured in 20% or 1% 02 for 24 h, then flow cytometric staining was performed (n=3, ns=non-significant).

FIG. 2

Hypoxia decreases the expression level of NK cell cytotoxicity related molecules. (a) Flow cytometric analysis of granzyme B and perforin expression in KHYG-1 (upper panel) and NK92 (lower panel) cells, respectively. KHYG-1 and NK92 were cultured in normoxic (20% 02) and hypoxic (1% 02) for 24 h, then intracellular staining was performed to analyze the expression of granzyme and perforin quantitatively. Left panel: Histogram overlays display representative examples of granzyme B, and perforin expression analyzed in normoxic and hypoxic cell samples compared to the fluorescence minus one (FMO) control; Right panel: statistical analysis of the flow cytometry data (n=3, * P<0.05, ** P<0.01). (b) Flow cytometric analysis of the intracellular level of IFN-γ in normoxic and hypoxic KHYG-1 and NK92 cells. Left panel: one of three representative flow cytometry results; Right panel: statistical analysis of the flow cytometry data (n=3, * P<0.05, ** P<0.01). (c) Flow cytometric analysis of the membrane staining of degranulation marker CD107a in normoxic and hypoxic KHYG-1 and NK92 cells. Left panel: one of three representative flow cytometry results; Right panel: statistical analysis of the flow cytometry data (n=3, *P<0.05, ** P<0.01). (d) Flow cytometric analysis of the membrane staining of activating receptor NKp30, NKp46, and NKG2D in normoxic and hypoxic KHYG-1 and NK92 cells. Left panel: one of three representative flow cytometry results; Right panel: statistical analysis of the flow cytometry data (n=3, * P<0.05, ** P<0.01).

FIG. 3

Hypoxia diminishes the phosphorylation level of ERK and STAT3. (a-c) Western blotting analysis shows the expression levels of the phosphorylated ERK and STAT3 in KHYG-1 (a), NK92 (b), and primary NK cells (c), respectively. (d) Inhibition of ERK and STAT3 decreases the expression of activating receptors on the NK cell surface. Representative flow cytometry results show the effects of STAT3 inhibitor cryptotanshinone (CPT) (upper panel) and ERK inhibitor U0126 (lower panel) on the expression of activating receptors on the NK cell surface. Left panel: one of three representative flow cytometry results; Right panel: statistical analysis of the flow cytometry data (n=3, * P<0.05, ** P<0.01). KHYG-1 and NK92 cells were treated with vehicle, 10 μM ERK inhibitor U0126, and 10 μM STAT3 inhibitor CPT for 24 h. (e) Inhibition of ERK and STAT3 decreases NK cell cytotoxicity. Statistical analysis showing the effects of ERK and STAT3 inhibition on NK cells cytotoxicity against K562 cells (n=3, * P<0.05, ** P<0.01). KHYG-1 cells were pretreated with 10 μM U0126 and 10 μM CPT for 6 h and then incubated with K562 at different E:T ratios for 4 h.

FIG. 4

Hypoxia activates SHP-1 and SHP-2 in NK cells. (a-c) Western blotting analysis shows SHP-1 and SHP-2 expression in normoxic (20% 02) and hypoxic (1% 02) KHYG-1 (a), NK92 (b) cells, and primary NK cells (c), respectively. (d) Western blotting analysis shows the effects of SHP-1 inhibitor TPI-1 on the phosphorylation of ERK and STAT3. Hypoxic KYHG-1 cells were pre-treated with 5 μM TPI-1 for 2 h, and then the cells were collected for western blotting analysis. (e) Flow cytometric analysis of the effects of TPI-1 on the NK cell cytotoxicity. Left panel: representative flow cytometry results of TPI-1 on the cytotoxicity of KHYG-1 cells. KHYG-1 cells were pretreated with 5 μM TPI-1 for 2 h, and then incubated with K562 cells at different E:T ratios for 4 h. Right panel: statistical analysis of the effects of TPI-1 on KHYG-1 cell cytotoxicity against K562 cells (n=3, * P<0.05).

FIG. 5

Inhibition of SHP-2 has no effect on NK cell cytotoxicity. (a) Western blotting shows the effects of SHP-2 inhibitor SHP099 on the phosphorylation of ERK and STAT3. Hypoxic KYHG-1 cells were pre-treated with 5 μM SHP099 for 2 h, and then the cells were collected for western blotting analysis. (b) Flow cytometric analysis shows the effect of SHP099 on the cytotoxicity of KHYG-1 cells. KHYG-1 cells were pretreated with 5 μM SHP099 for 2 h, then incubated with K562 cells at different E:T ratios for 4 h. (c) Statistical analysis of the effects of SHP099 on KHYG-1 cell cytotoxicity against K562 cells (n=3, ns=non-significant).

FIG. 6

The effects of gene silencing SHP-1 on ERK and STAT3 signaling as well as NK cell cytotoxicity. (a) Western blotting analysis of the SHP-1, ERK, and STAT3 expressions in siRNA-mediated knockdown of SHP-1 in KHYG-1 cells. (b) Statistical analysis of the effects of knocking down SHP-1 on NK cells cytotoxicity against K562 cells (n=3, * P<0.05). KHYG-1 cells were electroporated with 2 μg siRNA, and then cultured for 12-16 h in the RPMI 1640 growth medium containing IL-2. The electroporated cells were used for western blotting or killing assay as previously mentioned. (c) A schematic diagram shows how hypoxia impairs NK cell cytotoxicity in a SHP-1 dependent manner.

### Examples 1-3: Materials and Methods

Antibodies and Reagents

Antibodies for Western blotting against phospho-Stat3 (#4113), Stat3 (#12640), Phospho-p44/42 MAPK (ERK1/2) (#9106), p44/p42 MAPK (ERK1/2) (#9102), Phospho-SHP-1 (#8849), Phospho-SHP-2 (#5431), SHP-1 (#3759), SHP-2 (#3397), HIF-1α (#14179) and β-actin (#58169) were bought from Cell Signaling. Peroxidase-conjugated goat anti-rabbit IgG (#111-035-003) or goat anti-mouse IgG (#115-005-003) were bought from Jackson ImmunoResearch. For flow cytometry analysis, Alexa Fluor 647-labeled anti-human perforin (#563576) was purchased from BD Biosciences. FITC-labeled Annexin V (#640945), PE-labeled anti-human IFN-γ (#506506), anti-human/mouse granzyme B (#372207), APC-labeled anti-human NKp46 (#137607), anti-human NKp30 (#325209), anti-human NKG2D (#320808), anti-human CD2 (#300214), anti-human CD107a (#12-1079-42) antibodies, were purchased from Biolegend. CD3-labeled CD3 (#560365), PE-labeled CD56 (#555516) were bought from BD Biosciences. Sytox® Green Dead Cell Stain was bought from Molecular Probes (#S34860). STAT3 inhibitor Cryptotanshinone was bought from Selleck Chemicals (#35825-57-1). SHP-1 inhibitor TPI-1 (#HY-100463), SHP-2 inhibitor SHP-099 (#HY-100388) and ERK inhibitor U0126 (#HY-12031) were bought from MedChem Express.

Cell Lines and Cell Culture

The NK cell line KHYG-1 was cultured in RPMI-1640 (BasalMedium, #L210KJ) supplemented with 10% fetal bovine serum (FBS) (Biological Industries, #04-001-1ACS), 10 ng/mL human IL-2 (PeproTech, #200-02), 2 mM L-glutamine, 100 U/mL penicillin and 100 μg/mL streptomycin. The NK cell line NK92 was cultured in Dulbecco's modified Eagle's medium (DMEM) (BasalMedium, #L110KJ) supplemented with 10% FBS, 10% horse serum, 10 ng/mL IL-2, 2 mM glutamine, 100 U/mL penicillin and 100 μg/mL streptomycin. The human multiple myeloma cell line MM.1S and leukemia cell line K562 were grown in RPMI-1640 supplemented with 10% FBS, 2 mM glutamine, 100U/mL penicillin, and 100 μg/mL streptomycin. All cells were maintained at 37° C. in a humidified atmosphere containing 5% CO₂. Normoxic or hypoxic cell culture conditions were obtained by culturing cells in a sealed incubator flushed with a mixture of 20% O₂, 5% CO₂, and 75% N₂, or the mixture of 1% O₂, 5% CO₂, and 94% N₂, respectively.

Human Primary NK Cell Enrichment and Activation

Primary NK cells were isolated from peripheral blood mononuclear cells of healthy human donors through an immunomagnetic negative selection strategy (EasySep Human NK cell Isolation Kit, Stemcell Technologies) according to the manufacturer's protocol. Purity of the purified NK cell populations was determined by flow cytometry using fluorochrome-conjugated antibodies against CD3, CD56. For short-term activation, purified NK cells (>90% pure) were resuspended in RPMI-1640 supplemented with 10% FBS, 5% human serum, 2 mM glutamine, 100U/mL penicillin and 100 μg/mL streptomycin at a density of 3×10⁶ cells/ml and cultured overnight in the presence of IL-2 (10 ng/ml) under normoxic or hypoxic conditions as described above.

Flow Cytometry

The expression of NK cell cytotoxicity effector molecules and activating receptors was analyzed by flow cytometry. For membrane staining, 5×10⁵ cells were collected and washed with staining buffer (PBS containing 0.1% NaN₃ and 0.1% BSA) three times. The cells were then incubated for 30 min on ice, according to the instructions provided with the respective antibodies. After washing 3 times with cell stain buffer, the cells were resuspended in 300 μL staining buffer in the presence of Sytox Green or 7-AAD, which were used to gate out dead cells. Acquisition of 10,000 cells per reaction was performed using a CytoFLEX Cytometer (Beckman Coulter Life Sciences). Data were analyzed with Flowjo v7.6.2 (Tree Star). For intracellular staining, 5×10⁵ cells were collected and fixed with 1 mL 1% paraformaldehyde in PBS for 15 minutes at room temperature. After washing 3 times with cell stain buffer, the fixed cells were then resuspended in 2 mL permeabilization buffer (0.1% saponin in cell staining buffer) and incubated for 30 min at room temperature. The cells were collected again by centrifugation, stained with the antibody at an optimal working concentration in permeabilization buffer for 15 min on ice. After washing three times with permeabilization buffer, the cells were resuspended cells in 300 μL cell staining buffer for final flow cytometric analysis.

CD107a Degranulation Assay

Degranulation of cytotoxic contents from NK cells was measured by analysis of the degranulation marker CD107a by flow cytometry. Briefly, NK cells and tumour cells were individually pre-incubated for 14-16 h at 20% or 1% 02 and after that combined at 1:1 (E:T) ratio at either 20% or 1% 02 in 24-well plate. 5 μL of APC labeled anti-CD107a was added to the wells within 5-10 minutes after combining NK and tumour cells. Subsequently, Monensin and GolgiPlug (1:1000 dilution; BD Biosciences) were added. After a total incubation time of 4 h, the plate was placed on ice to stop the reaction. Cells were then harvested and analyzed using flow cytometry.

Flow Cytometric Cytotoxicity Assay

Prior to the assay, NK cells and tumour cells were individually pre-incubated for 24 h at 5% CO₂ with 20% or 1% 02 first. NK and target cells were then incubated under comparable conditions in different E:T ratios in a 24 well plate. After the 4 h incubation, samples were harvested and washed followed by a combinational staining with CD2-APC, Annexin V-FITC as well as Sytox Green, in which CD2 was used to distinguish effector from target cells, and target cell death was detected with Annexin V-FITC and Sytox Green. A minimum of 10,000 target events were collected per sample, and the results were analyzed using Flowjo v7.6.2.

Western Blotting

For western blotting, treated and untreated KHYG-1 and NK92 cells were lysed in buffer containing 50 mM Tris, 150 mM NaCl, 1% Triton X-100, 1% sodium deoxycholate, 0.1% SDS and protease inhibitors on ice for 30 min. Lysates were centrifuged at 12,000 rpm for 15 min, and supernatants were collected. Protein concentration was determined by the BCA protein assay kit (HEART Biotech, #WB003). Equal amounts of protein were loaded and separated on sodium dodecyl sulfate-polyacrylamide gel electrophoresis gel, and transferred onto a PVDF membrane (Millipore, #IPVH00010). After blocking for 1 h with 5% non-fat milk in PBS with 0.1% Tween-20 at room temperature, the membrane was incubated with primary antibody at 4° C. overnight. Immunoblots were visualized using HRP-conjugated secondary antibodies and the ECL Western Blot Detection kit (Phygene Life Sciences, #PH0353).

siRNA-Mediated Gene Silencing in NK Cells

Prior to siRNA transfection, KHYG-1 cells were washed in pre-warmed Opti-MEM medium (Life Technologies, Carlsbad, Calif., USA) and resuspended in the same medium. Then, 10⁶ cells were electroporated with 2 μg of siRNA in 100 μL Opti-MEM medium in 0.2 cm cuvettes with an electroporator CUY21EDIT (BEX Co. Ltd, Japan). The electroporation program was set as follows: PpV=200V, Pp on 10 ms, Pp off 10 ms, PdV=25V, Pd on 50 ms, Pd off 50 ms; Pd N=10, capacity=940 μF, and exponential decay wave type. Following electroporation, cells were resuspended in 2 mL complete media and cultured in hypoxic condition (1% O₂). 16-24 h after electroporation, the cells were used for western blotting or killing assay. Transfection efficiency and viability were analyzed by flow cytometry 2-6 h after electroporation by quantitatively measuring the expression of fluorescein isothiocyanate (FITC)-labeled siRNA or 7-AAD. SHP-1 mRNA was silenced by using a gene-specific siRNA pool from GenePharma.

Statistical Analysis

Statistical analyses were performed using the Prism software package 5.0 (GraphPad Software, San Diego, Calif., USA). Data are expressed as the mean±SEM of at least three independent experiments. Statistical significance was evaluated by two-tailed paired Student's t-test. P<0.05 (*), P<0.01(**), or P<0.001(***) was considered statistically significant.

### Example 1—Reduced Cytotoxicity of Hypoxic NK Cells

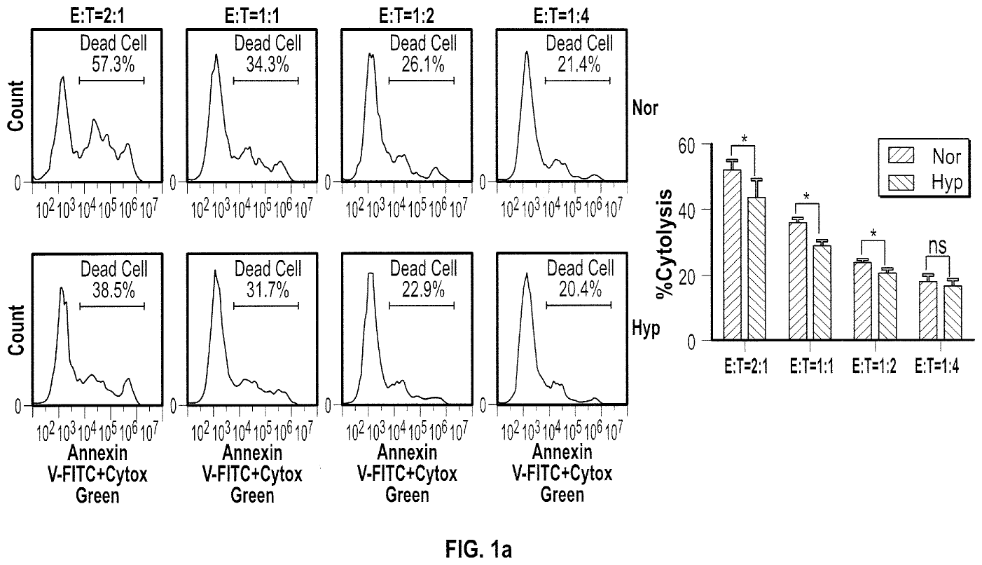

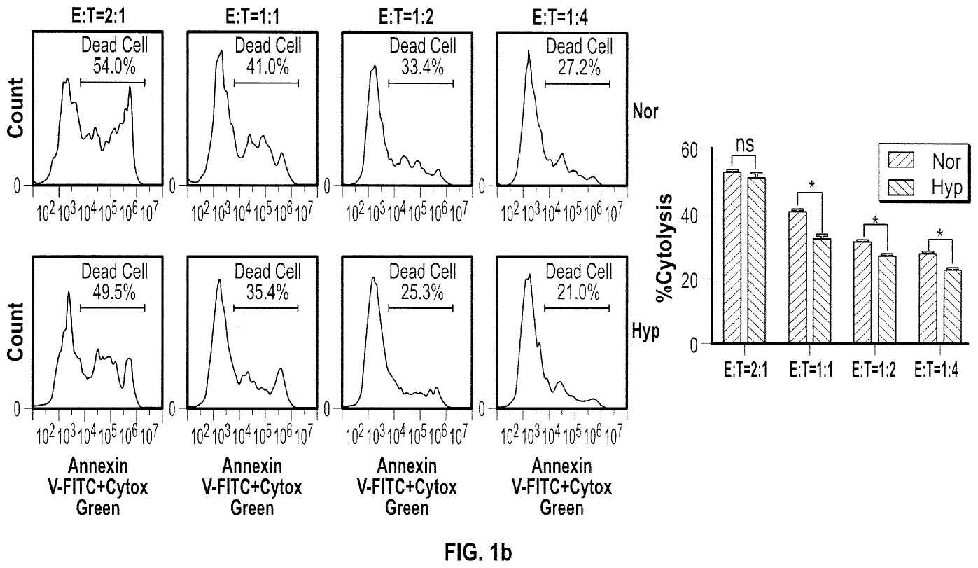

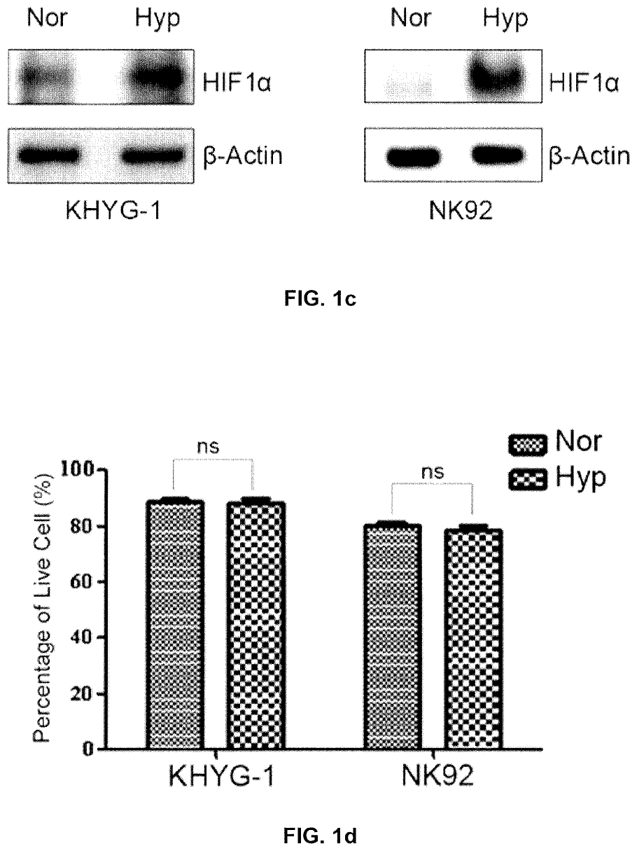

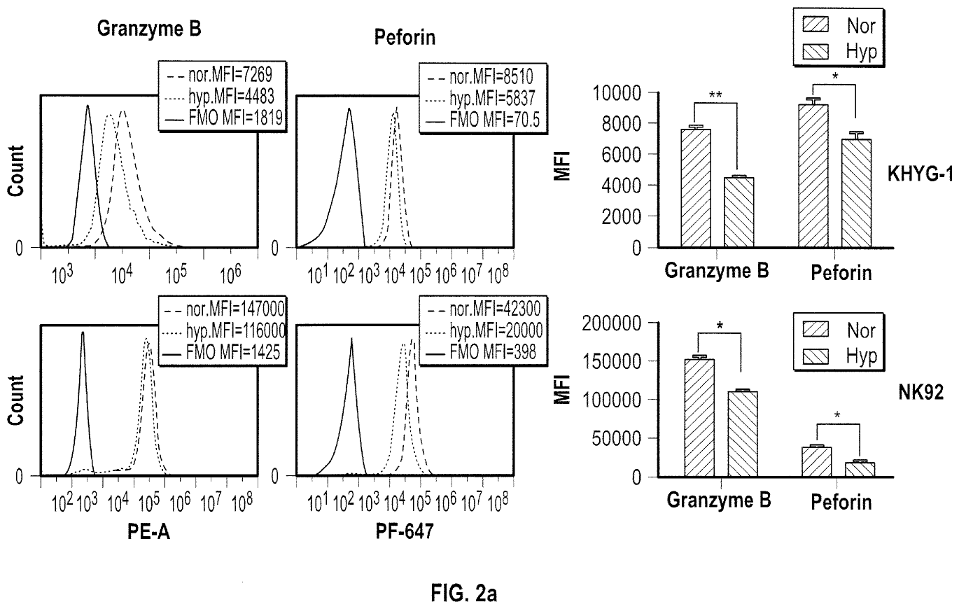

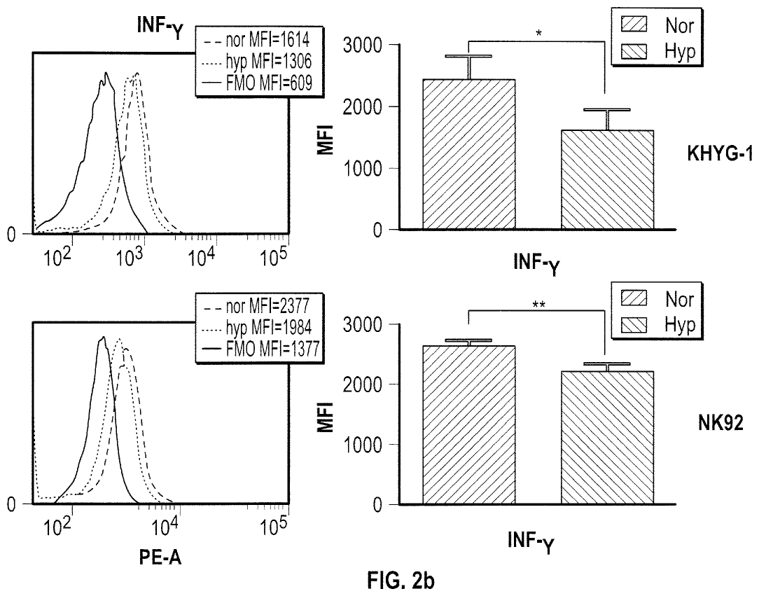

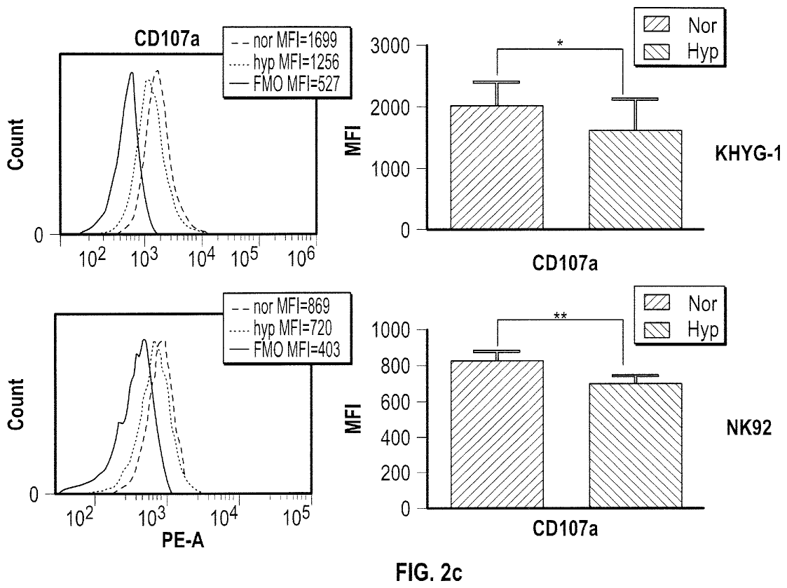

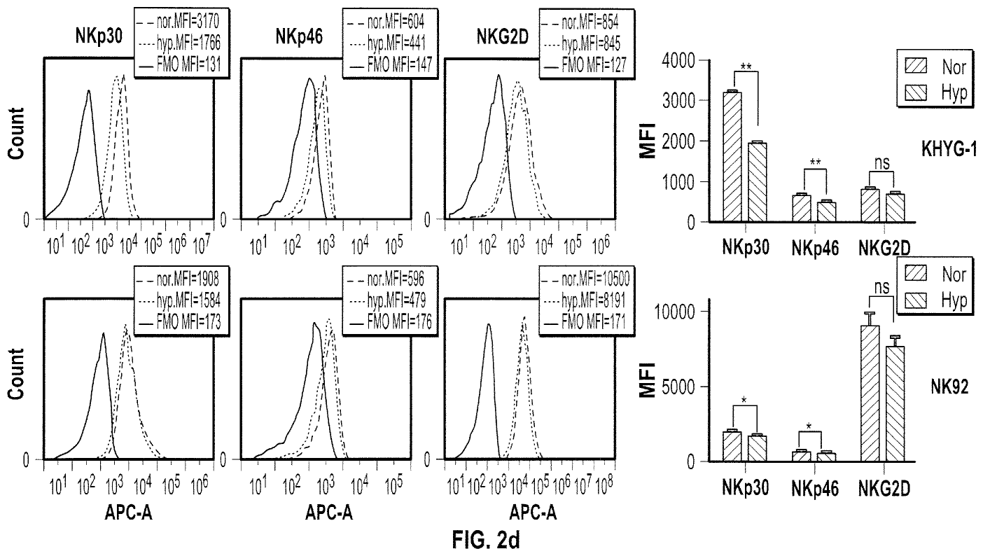

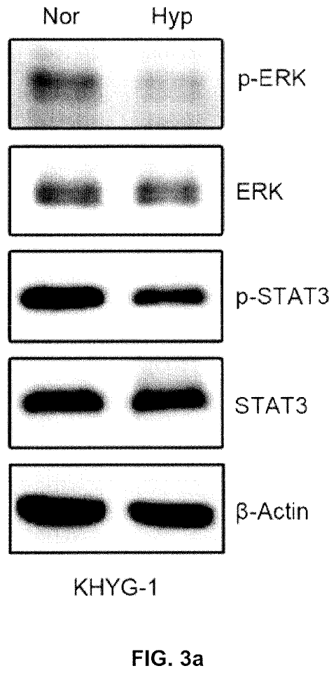

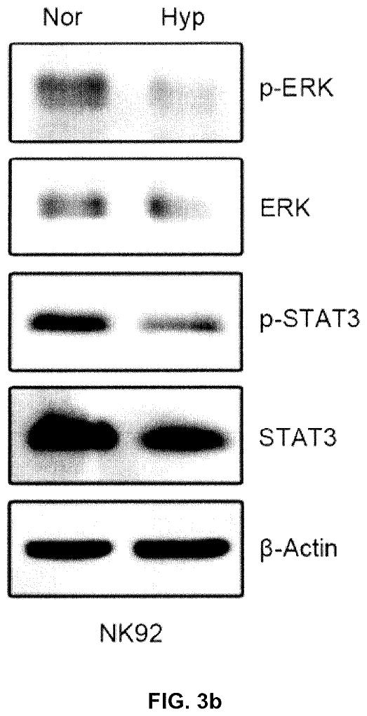

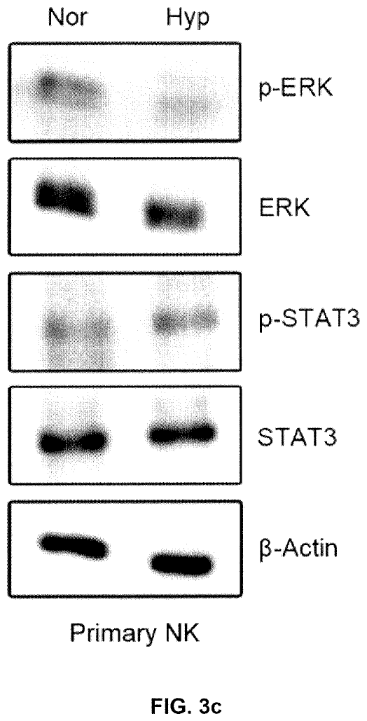

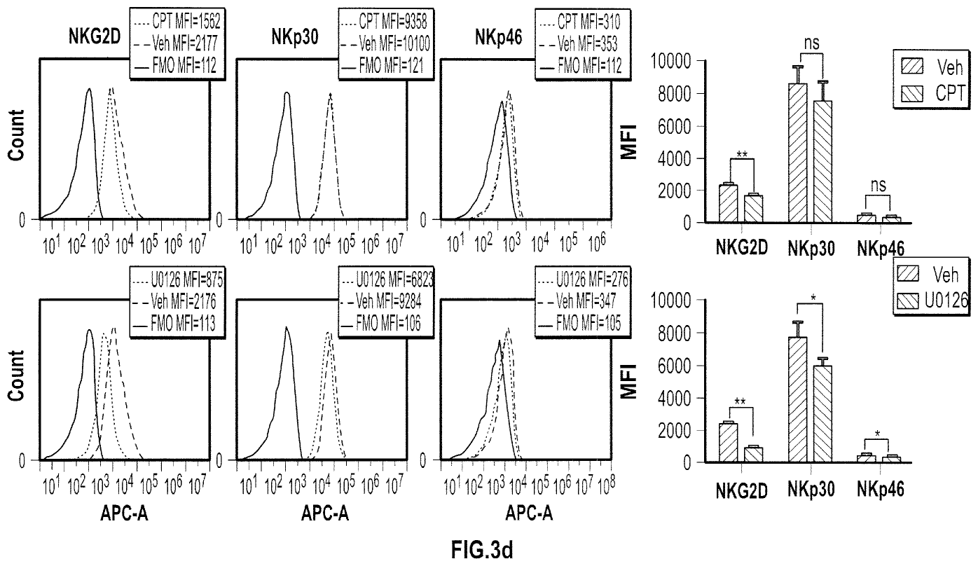

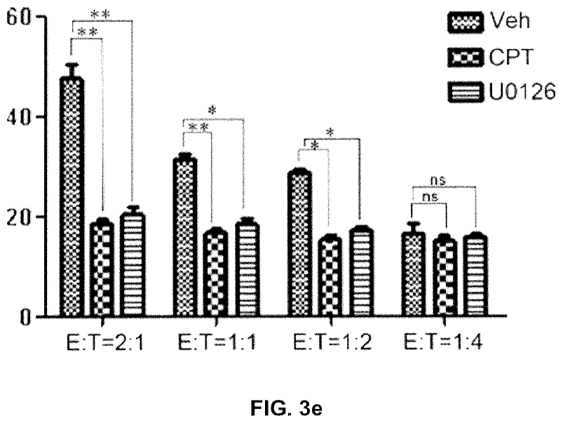

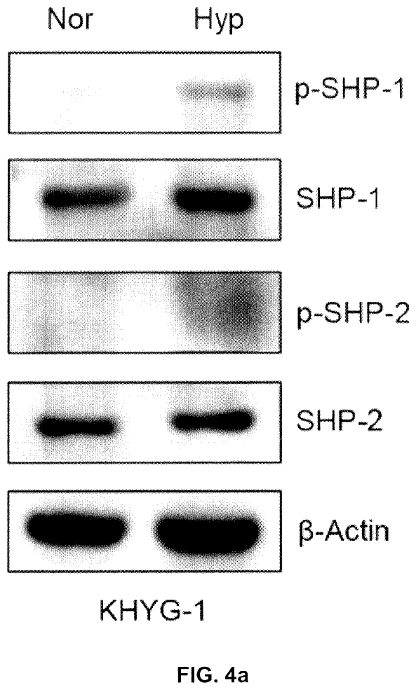

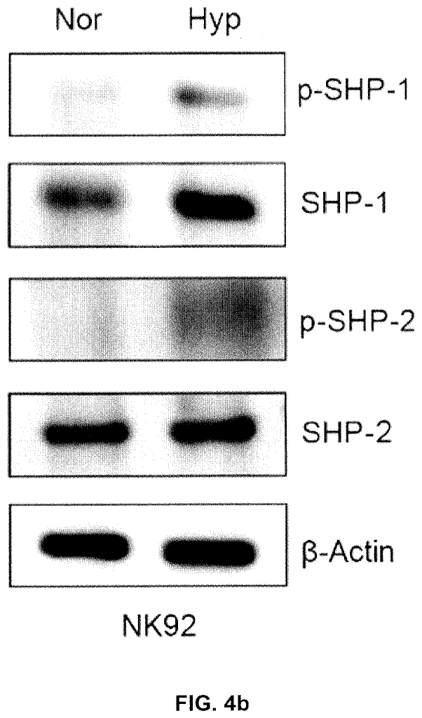

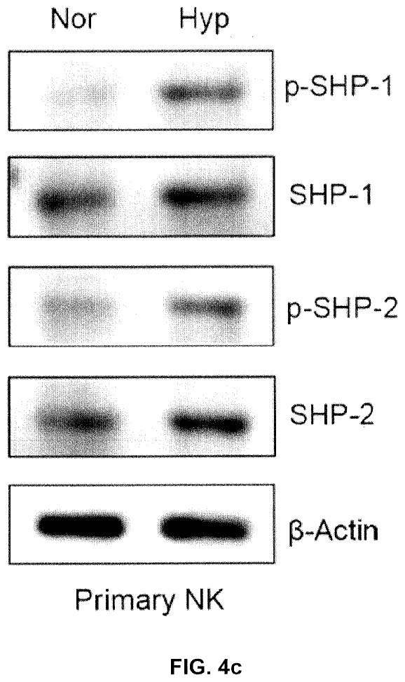

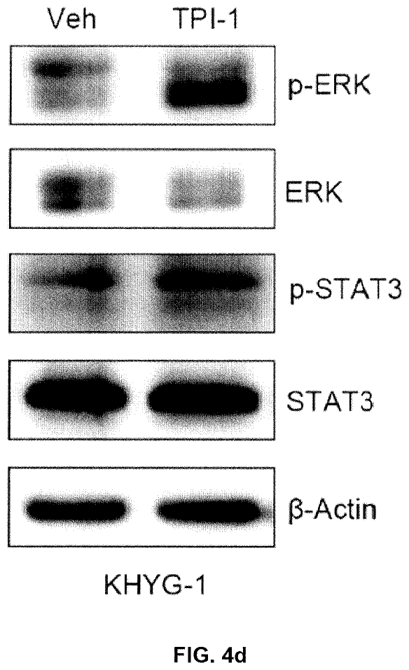

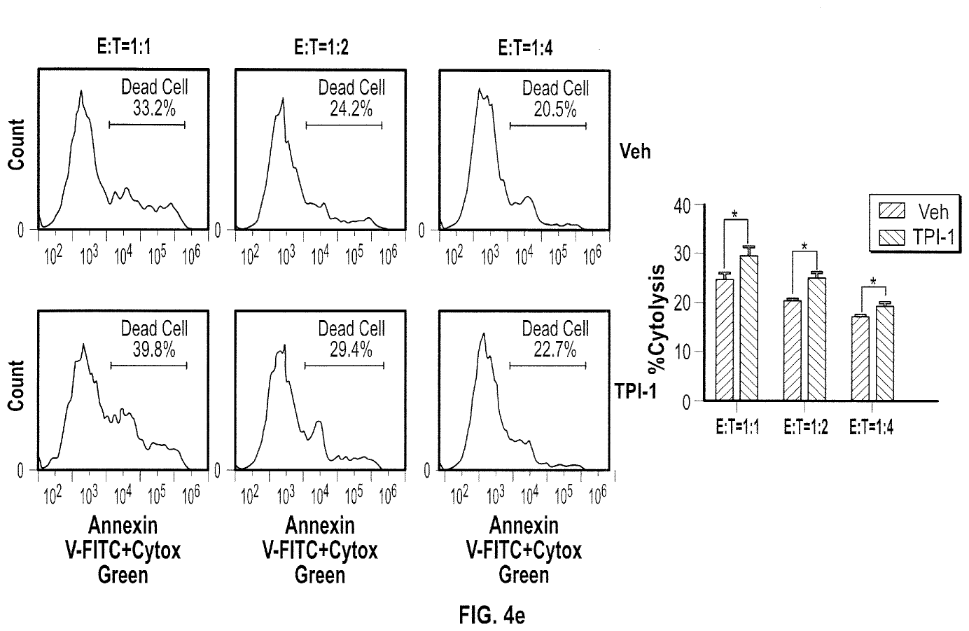

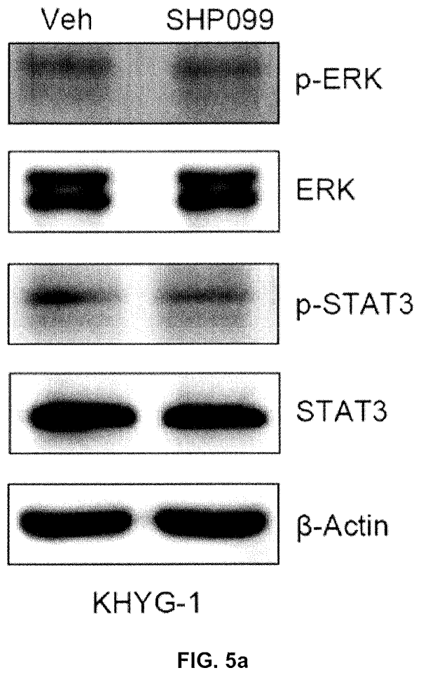

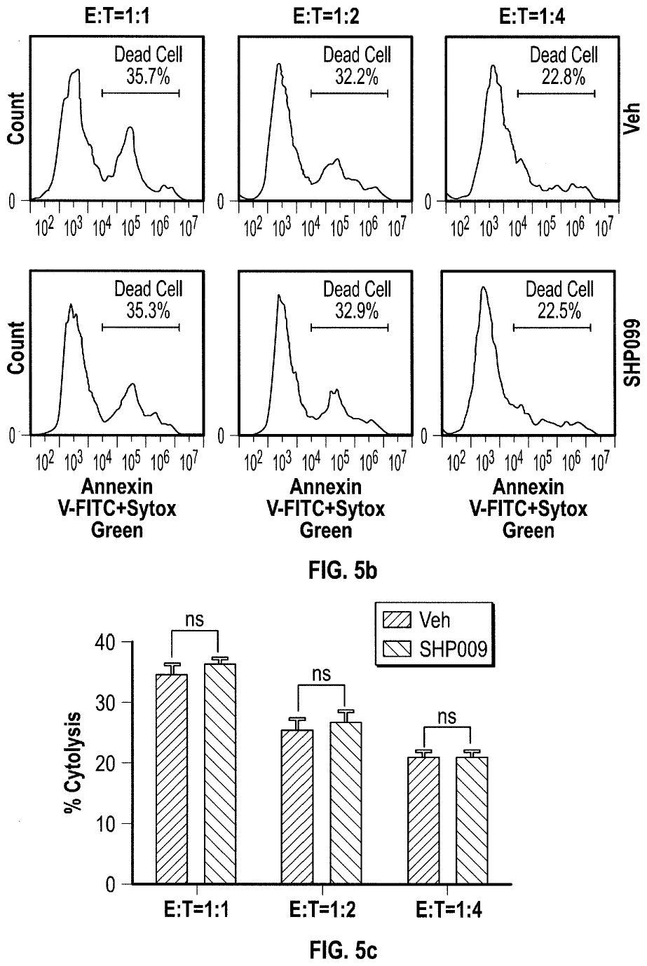

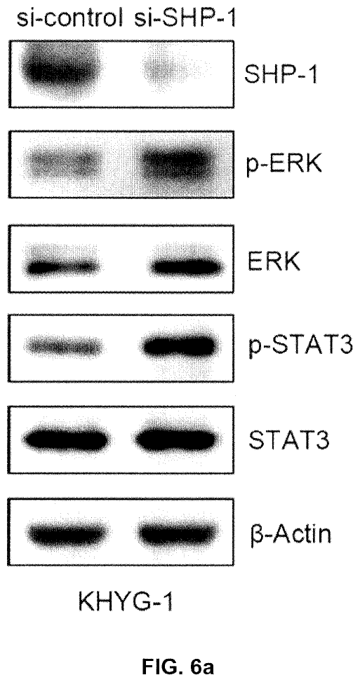

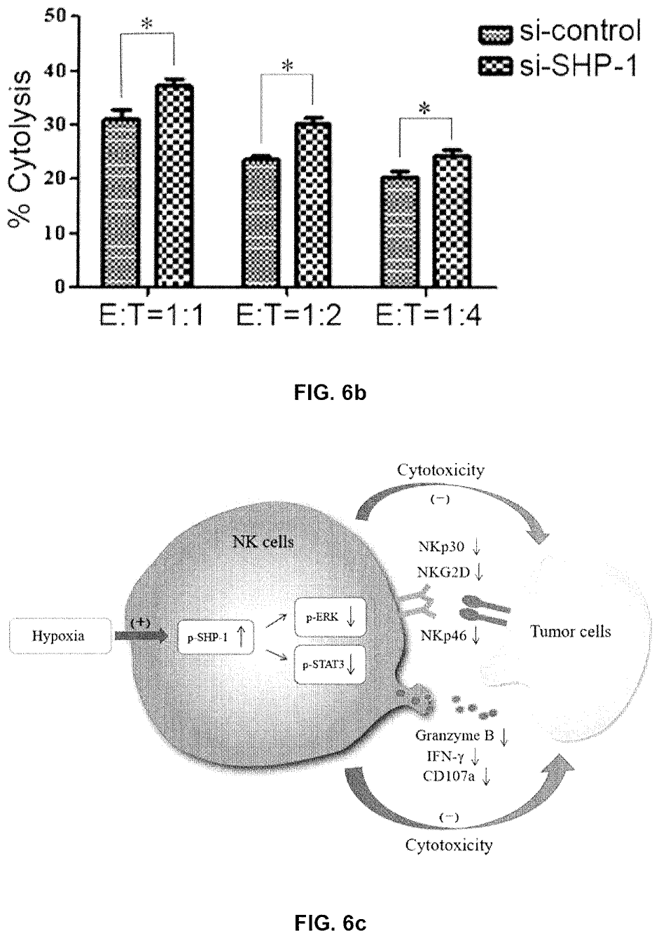

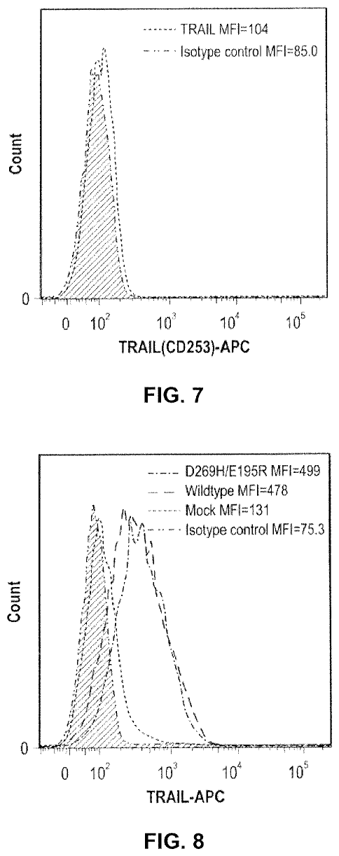

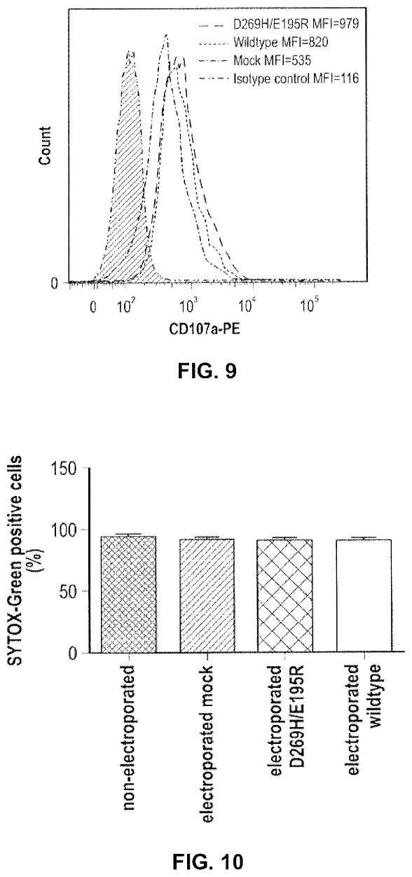

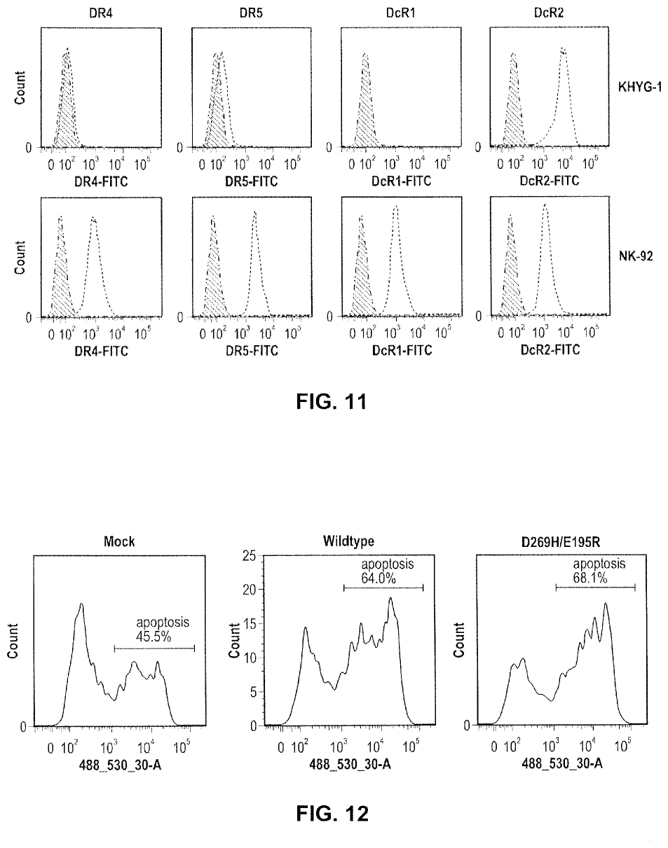

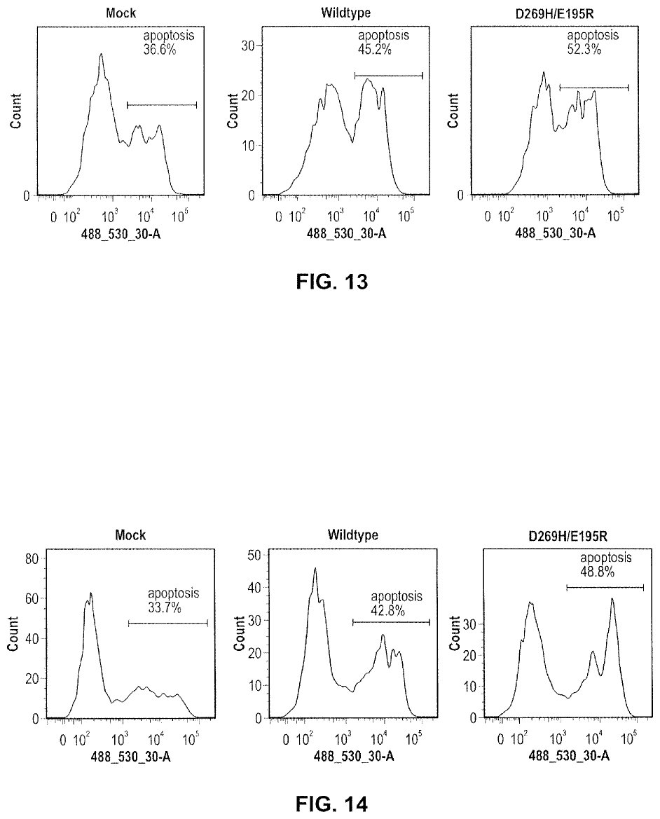

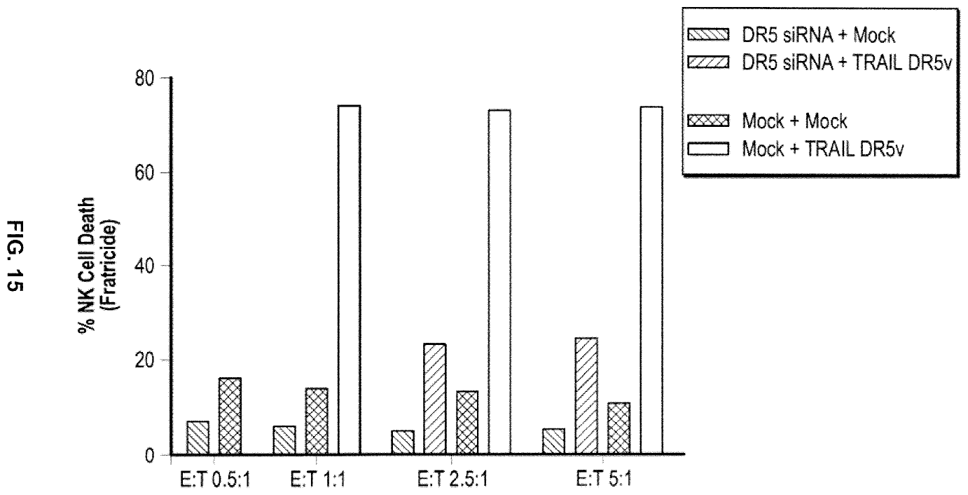

The effect of hypoxia on NK cell-mediated lysis of tumour cells was investigated. KHYG-1 cells were cultured in the presence of IL-2 under hypoxic (1% O₂) or normoxic (20% O₂) conditions for 24 hours, and subsequently the cells were incubated with cancer cell lines K562 or MM.1S at different effector:target (E:T) ratios for 4 hours, in order to evaluate cytotoxicity by flow cytometry. As shown in FIG. 1a and FIG. 1b, NK cell cytotoxicity was significantly decreased in hypoxic conditions.

Western blotting revealed a significant accumulation of the hypoxia marker HIF-1α in hypoxic NK cells (KHYG-1 and NK-92), whereas HIF-1a was only weakly expressed in normoxic NK cells—see FIG. 1c.

The possibility that the decreased cytotoxicity of hypoxic NK cells was caused by reduced NK cell viability was eliminated, as there was no significant difference in NK cell death between the hypoxic and normoxic samples (FIG. 1d).

To further investigate how hypoxia reduces NK cell cytotoxicity, the expression levels of the cytotoxic effectors granzyme B and perforin were measured. As shown in FIG. 2a, hypoxia led to decreased secretion of both granzyme B and perforin. Additionally, a reduced expression of the cytokine IFN-γ was observed in hypoxic NK cells, when compared to normoxic NK cells (FIG. 2b). The degranulation marker CD107a was also diminished by hypoxia (FIG. 2c).

Surface expression of the activating receptors NKp46, NKp30 and NKG2D was measured by flow cytometry on both normoxic NK cells and hypoxic NK cells. As shown in FIG. 2d, hypoxic conditions decreased the expression of these activating receptors on the NK cell surface.

### Example 2—Hypoxic Attenuation of ERK and STAT3-Mediated NK Cell Activation

It is known that intracellular signals activating NK cell cytotoxicity are propagated primarily through protein phosphorylation of extracellular signal-regulated kinase (ERK) and signal transducer and activator of transcription 3 (STAT3). The role of hypoxia in the activation of ERK and STAT3 was therefore investigated.

It was revealed that hypoxia markedly decreased the phosphorylation level at the tyrosine sites of ERK and STAT3 in KHYG-1 cells, NK-92 cells and primary NK cells (FIGS. 3a, 3b and 3c).

To further validate the effects of ERK and STAT3 phophorylation on NK cell cytotoxicity under hypoxic conditions, specific small molecule inhibitors U0126 and cryptotanshinone were used to block ERK and STAT3 signaling, respectively. As shown in FIG. 3d, inhibition of ERK and STAT3 significantly reduced the expression of activating receptors NKp30, NKp46 and NKG2D (KHYG-1) and NKp46 and NKG2D (NK-92). Moreover, inhibition of ERK or STAT3 resulted in significantly impaired NK cell cytotoxicity against cancer cells (FIG. 3e).

Cell surface receptors harboring intracytoplasmic tyrosine-based activation motifs (ITAMs) or intracytoplasmic tyrosine-based inhibitory motifs (ITIMs) are often phosphorylated by Src family protein tyrosine kinase (PTK), which in turn creates docking sites for the protein tyrosine phosphatases SHP-1 and SHP-2. The involvement of SHP-1 and SHP-2 in the decrease of ERK and STAT3 phosphorylation by hypoxia was therefore investigated. As shown in FIGS. 4a, 4b and 4c, hypoxia induced a significant increase in the phosphorylation of SHP-1 and SHP-2 in the two NK cell lines and in primary NK cells. When using a specific SHP-1 inhibitor, TPI-1, it was observed that TPI-1 could reverse the decrease of the phosphorylation of both ERK and STAT3 (FIG. 4d). Moreover, it was also observed that pretreatment with the p-SHP1 inhibitor TPI-1 could restore NK cell cytotoxicity under hypoxia (FIG. 4e). The same effects were not observed when using the specific SHP-2 inhibitor SHP099, which had no effect on the phosphorylation levels of ERK and STAT3, or NK cell cytotoxicity (FIGS. 5a, 5b and 5c).

It was thus concluded that a hypoxia-induced decrease in the phosphorylation level of STAT3 and ERK was mediated by the activation of the protein tyrosine phosphatase SHP-1, as opposed to SHP-2

### Example 3—Knockdown of SHP-1 Restores NK Cell Cytotoxicity in Hypoxia

To further validate the role of SHP-1 in regulating NK cell cytotoxicity, SHP-1 gene expression was silenced in KHYG-1 cells. It was confirmed that knockdown of SHP-1 could increase the phosphorylation level of ERK and STAT3 under hypoxia (FIG. 6a). Moreover, it was confirmed that NK cells with SHP-1 silenced showed greater cytotoxicity against K562 cells than control NK cells under hypoxic conditions (FIG. 6b).

### Example 4—Knock-In of CD19 CARs in Primary NK Cells

The anti-CD19-ζ, anti-CD19-BB-ζ, and anti-CD19-truncated (control) plasmids used have been described previously (Imai et al. 2004. Leukemia. 18(4):676-84). The cDNA encoding the intracellular domains of human DAP10 and 4-1BB ligand (4-1BBL), and interleukin-15 (IL-15) with long signal peptide were sub-cloned by polymerase chain reaction (PCR) with a human spleen cDNA library used as a template. An anti-CD19-DAP10 plasmid was constructed by replacing the sequence encoding CD3 with that encoding DAP10, using the splicing by overlapping extension by PCR (SOE-PCR) method. The cDNA encoding the signal peptide of CD8α, the mature peptide of IL-15 and the transmembrane domain of CD8α were assembled by SOE-PCR to encode a “membrane-bound” form of IL-15. The resulting expression cassettes were sub-cloned into EcoRI and Xhol sites of murine stem-cell virus-internal ribosome entry site-green fluorescent protein (MSCV-IRES-GFP).

The RD114-pseudotyped retrovirus was generated as described previously (Imai et al. 2004. Leukemia. 18(4):676-84). A calcium phosphate DNA precipitation was used to transfect 293T cells with anti-CD19-ζ, anti-CD19-DAP10, anti-CD19-BB-ζ, or anti-CD19-truncated; pEQ-PAM3(-E); and pRDF. Conditioned medium containing retrovirus was harvested at 48 hours and 72 hours after transfection, immediately frozen in dry ice, and stored at −80° C. until use.

K562 cells were transduced with the construct encoding the “membrane-bound” form of IL-15. Cells were cloned by limiting dilution, and a single-cell clone with high expression of GFP and surface IL-15 (K562-mb15) was expanded. This clone was subsequently transduced with human 4-1BBL (K562-mb15-41BBL). K562 cells expressing wildtype IL-15 (K562-wt15) or 4-1BBL (K562-41BBL) were produced by a similar procedure. Peripheral blood mononuclear cells (1.5×10⁶) were incubated in a 24-well tissue-culture plate with or without 10⁶ K562-derivative stimulator cells in the presence of 10 IU/mL human IL-2 in RPMI 1640 and 10% FCS.

Mononuclear cells stimulated with K562-mb15-41BBL were transduced with retroviruses, as described previously (Imai et al. 2004. Leukemia. 18(4):676-84). Briefly, 14 mL polypropylene centrifuge tubes were coated with human fibronectin (100 μg/mL) or RetroNectin (50 μg/mL). Pre-stimulated cells (2×10⁵) were re-suspended in the tubes in 2-3 mL virus-conditioned medium with Polybrene (4 μg/mL) and centrifuged at 2400 g for 2 hours (centrifugation was omitted when RetroNectin was used). The multiplicity of infection (4-6) was identical in each experiment comparing the activity of different CARs. After centrifugation, cells were left undisturbed for 24 hours in a humidified incubator at 37° C., 5% CO₂. The transduction procedure was repeated on 2 successive days. After a second transduction, the cells were re-stimulated with K562-mb15-41BBL in the presence of 10 IU/mL IL-2. Cells were maintained in RPMI 1640, 10% FCS and 10 IU/mL IL-2.

Transduced NK cells were stained with goat anti-mouse (Fab)₂ polyclonal antibody conjugated with biotin followed by streptavidin conjugated to peridinin chlorophyll protein. For Western blotting, cells were lysed in RIPA buffer (PBS, 1% Triton-X100, 0.5% sodium deoxycholate, 0.1% sodium dodecyl sulfate [SDS]) containing 3 μg/mL pepstatin, 3 μg/mL leupeptin, 1 mM phenylmethylsulfonyl fluoride (PMSF), 2 mM ethylenediaminetetraacetic acid (EDTA) and 5 μg/mL aprotinin. Centrifuged lysate supernatants were boiled with an equal volume of loading buffer, with or without 0.1 M dithiothreitol (DTT), and then separated by SDS-polyacrylamide gel electrophoresis (SDS-PAGE) on a precast 10-20% gradient acrylamide gel. The proteins were transferred to a polyvinylidene fluoride (PVDF) membrane which was incubated with primary mouse anti-human CD3 monoclonal antibody (clone 8D3). Membranes were then washed, incubated with a goat anti-mouse IgG horseradish peroxidase-conjugated second antibody and developed using an enhanced chemiluminescence system.

The following antibodies were used for immunophenotypic characterization of expanded and transduced cells: anti-CD3 conjugated to fluorescein isothiocyanate (FITC), to PerCP or to energy-coupled dye (ECD); anti-CD10 conjugated to phycoerythrin (PE); anti-CD19 PE; anti-CD22 PE; anti-CD56 FITC, PE or allophycocyanin (APC); anti-CD16 CyChrome; and anti-CD25 PE. Surface expression of KIR and NK activation molecules was determined with specific antibodies conjugated to FITC or PE, as described previously (Leung et al. 2004. Journal of Immunology. 172:644-650). Antibody staining was detected with a FACScan or an LSR II flow cytometer.

### Example 5—Knock-In of TRAIL/TRAIL Variant in NK Cells

KHYG-1 cells were transfected with both TRAIL and TRAIL variant, in order to assess their viability and ability to kill cancer cells following transfection.

The TRAIL variant used is that described in WO 2009/077857. It is encoded by the wildtype TRAIL gene containing the D269H/E195R mutation. This mutation significantly increases the affinity of the TRAIL variant for DR5, whilst reducing the affinity for both decoy receptors (DcR1 and DcR2).

Baseline TRAIL Expression

Baseline TRAIL (CD253) expression in KHYG-1 cells was assayed using flow cytometry.

Mouse anti-human CD253-APC (Biolegend catalog number: 308210) and isotype control (Biolegend catalog number: 400122) were used to stain cell samples and were analyzed on a BD FACS Canto II flow cytometer.

KHYG-1 cells were cultured in RPMI 1640 medium containing 10% FBS, 2 mM L-glutamine, penicillin (100 U/mL)/streptomycin (100 mg/mL) and IL-2 (10 ng/mL). 0.5-1.0×10⁶ cells/test were collected by centrifugation (1500 rpm×5 minutes) and the supernatant was aspirated. The cells (single cell suspension) were washed with 4 mL ice cold FACS Buffer (PBS, 0.5-1% BSA, 0.1% NaN₃ sodium azide). The cells were re-suspended in 100 μL ice cold FACS Buffer, add 5 uL antibody was added to each tube and incubated for 30 minutes on ice. The cells were washed 3 times by centrifugation at 1500 rpm for 5 minutes. The cells were then re-suspended in 500 μL ice cold FACS Buffer and temporarily kept in the dark on ice.

The cells were subsequently analyzed on the flow cytometer (BD FACS Canto II) and the generated data were processed using FlowJo 7.6.2 software.

As can be seen in FIG. 7, FACS analysis showed weak baseline expression of TRAIL on the KHYG-1 cell surface.

TRAIL/TRAIL Variant Knock-In by Electroporation

Wildtype TRAIL mRNA and TRAIL variant (D269H/195R) mRNA was synthesized by TriLink BioTechnologies, aliquoted and stored as −80° C. Mouse anti-human CD253-APC (Biolegend catalog number: 308210) and isotype control (Biolegend catalog number: 400122), and Mouse anti-human CD107a-PE (eBioscience catalog number: 12-1079-42) and isotype control (eBioscience catalog number: 12-4714) antibodies were used to stain cell samples and were analyzed on a BD FACS Canto II flow cytometer. DNA dye SYTOX-Green (Life Technologies catalog number: S7020; 5 mM Solution in DMSO) was used. To achieve transfection efficiencies of up to 90% with high cell viability in KHYG-1 cells with the Nucleofector™ Device (Nucleofector II, Lonza), a Nucleofector™ Kit T from Lonza was used. Antibiotics-free RPMI 1640 containing 10% FBS, L-glutamine (2 mM) and IL-2 (10 ng/mL) was used for post-Nucleofection culture.

KHYG-1 and NK-92 cells were passaged one or two days before Nucleofection, as the cells must be in the logarithmic growth phase. The Nucleofector solution was pre-warmed to room temperature (100 μl per sample), along with an aliquot of culture medium containing serum and supplements at 37° C. in a 50 mL tube. 6-well plates were prepared by filling with 1.5 mL culture medium containing serum and supplements and pre-incubated in a humidified 37° C./5% CO₂ incubator. An aliquot of cell culture was prepared and the cells counted to determine the cell density. The required number of cells was centrifuged at 1500 rpm for 5 min, before discarding the supernatant completely. The cell pellet was re-suspended in room temperature Nucleofector Solution to a final concentration of 2×10⁶ cells/100p1 (maximum time in suspension=20 minutes). 100 μl cell suspension was mixed with 10 μg mRNA (volume of RNA <10 μL). The sample was transferred into an Amaxa-certified cuvette (making sure the sample covered the bottom of the cuvette and avoiding air bubbles). The appropriate Nucleofector program was selected (i.e. U-001 for KHYG-1 cells). The cuvettes were then inserted into the cuvette holder. 500 μl pre-warmed culture medium was added to the cuvette and the sample transferred into a prepared 6-well plate immediately after the program had finished, in order to avoid damage to the cells. The cells were incubated in a humidified 37° C./5% CO₂ incubator. Flow cytometric analysis and cytotoxicity assays were performed 12-16 hours after electroporation. Flow cytometry staining was carried out as above.

As can be seen in FIGS. 8 and 9, expression of TRAIL/TRAIL variant and CD107a (NK activation marker) increased post-transfection, confirming the successful knock-in of the TRAIL genes into KHYG-1 cells.

FIG. 10 provides evidence of KHYG-1 cell viability before and after transfection via electroporation. It can be seen that no statistically significant differences in cell viability are observed following transfection of the cells with TRAIL/TRAIL variant, confirming that the expression of wildtype or variant TRAIL is not toxic to the cells. This observation contradicts corresponding findings in NK-92 cells, which suggest the TRAIL variant gene knock-in is toxic to the cells (data not shown). Nevertheless, this is likely explained by the relatively high expression levels of TRAIL receptors DR4 and DR5 on the NK-92 cell surface (see FIG. 11).

Effects of TRAIL/TRAIL Variant on KHYG-1 Cell Cytotoxicity

Mouse anti-human CD2-APC antibody (BD Pharmingen catalog number: 560642) was used. Annexin V-FITC antibody (ImmunoTools catalog number: 31490013) was used. DNA dye SYTOX-Green (Life Technologies catalog number: S7020) was used. A 24-well cell culture plate (SARSTEDT AG catalog number: 83.3922) was used. Myelogenous leukemia cell line K562, multiple myeloma cell line RPMI8226 and MM1.S were used as target cells. K562, RPMI8226, MM1.S were cultured in RPMI 1640 medium containing 10% FBS, 2 mM L-glutamine and penicillin (100 U/mL)/streptomycin (100 mg/mL).

As explained above, KHYG-1 cells were transfected with TRAIL/TRAIL variant.

The target cells were washed and pelleted via centrifugation at 1500 rpm for 5 minutes. Transfected KHYG-1 cells were diluted to 0.5×10⁶/mL. The target cell density was then adjusted in pre-warmed RPMI 1640 medium, in order to produce effector:target (E:T) ratios of 1:1.

0.5 mL KHYG-1 cells and 0.5 mL target cells were then mixed in a 24-well culture plate and placed in a humidified 37° C./5% CO₂ incubator for 12 hours. Flow cytometric analysis was then used to assay KHYG-1 cell cytotoxicity; co-cultured cells (at different time points) were washed and then stained with CD2-APC antibody (5 μL/test), Annexin V-FITC (5 μL/test) and SYTOX-Green (5 μL/test) using Annexin V binding buffer.

Data were further analyzed using FlowJo 7.6.2 software. CD2-positive and CD2-negative gates were set, which represent KHYG-1 cell and target cell populations, respectively. The Annexin V-FITC and SYTOX-Green positive cells in the CD2-negative population were then analyzed for TRAIL-induced apoptosis.

FIGS. 12, 13 and 14 show the effects of both KHYG-1 cells expressing TRAIL or TRAIL variant on apoptosis for the three target cell lines: K562, RPMI8226 and MM1.S, respectively. It is apparent for all target cell populations that TRAIL expression on KHYG-1 cells increased the level of apoptosis, when compared to normal KHYG-1 cells (not transfected with TRAIL). Moreover, TRAIL variant expression on KHYG-1 cells further increased apoptosis in all target cell lines, when compared to KHYG-1 cells transfected with wildtype TRAIL.

### Example 6—Knock-In of CD19 CARs and TRAIL Variants in Primary NK Cells

Anti-CD19-CD28(TM)-CD3ζ, anti-CD19-41BB(TM)-CD3ζ, and anti-CD19-truncated (control) plasmids were used. The cDNA encoding the CD19 scFv, with transmembrane domains of human CD3, CD28 or 4-1 BB ligand (4-1 BBL), and with intracellular domains of CD3 were used as a template mRNA. The gene cassette containing the combination of CD19 CAR and TRAIL variant was synthesized as mRNA. CD19 CAR and high affinity TRAIL DR5 variant was delivered to the NK cells as two separate in vitro synthesized mRNAs at the same time.

The TRAIL variant used is that described in WO 2009/077857. It is encoded by the wildtype TRAIL gene containing the D269H/E195R mutation. This mutation significantly increases the affinity of the TRAIL variant for DR5, whilst reducing the affinity for both decoy receptors (DcR1 and DcR2).

Electroporated NK cells were stained with goat anti-mouse (Fab)₂ polyclonal antibody conjugated with biotin followed by streptavidin conjugated to PE or FITC flurophore. For Western blotting, cells were lysed in RIPA buffer (PBS, 1% Triton-X100, 0.5% sodium deoxycholate, 0.1% sodium dodecyl sulfate [SDS]) containing 3 μg/mL pepstatin, 3 μg/mL leupeptin, 1 mM phenylmethylsulfonyl fluoride (PMSF), 2 mM ethylenediaminetetraacetic acid (EDTA) and 5 μg/mL aprotinin. Centrifuged lysate supernatants were boiled with an equal volume of loading buffer, with or without 0.1 M dithiothreitol (DTT), and then separated by SDS-polyacrylamide gel electrophoresis (SDS-PAGE) on a precast 10-20% gradient acrylamide gel. The proteins were transferred to a polyvinylidene fluoride (PVDF) membrane which was incubated with primary mouse anti-human CD3 monoclonal antibody (clone 8D3). Membranes were then washed, incubated with a goat anti-mouse IgG horseradish peroxidase-conjugated second antibody and developed using an enhanced chemiluminescence system.

The following antibodies were used for immunophenotypic characterization of expanded and transduced cells: anti-CD3 conjugated to fluorescein isothiocyanate (FITC), to PerCP or to energy-coupled dye (ECD); anti-CD10 conjugated to phycoerythrin (PE); anti-CD19 PE; anti-CD22 PE; anti-CD56 FITC, PE or allophycocyanin (APC); anti-CD16 CyChrome; and anti-CD25 PE. Surface expression of KIR and NK activation molecules was determined with specific antibodies conjugated to FITC or PE, as described previously (Leung et al. 2004. Journal of Immunology. 172:644-650). Antibody staining was detected with a FACS Canto II flow cytometer.

Baseline TRAIL (CD253) expression in naive or expanded NK cells was assayed using flow cytometry.

Mouse anti-human CD253-APC (Biolegend catalog number: 308210) and isotype control (Biolegend catalog number: 400122) were used to stain cell samples and were analyzed on a BD FACS Canto II flow cytometer.

Primary NK cells were cultured in Miltenyi's NK cell expansion medium containing 10% human AB serum, penicillin (100 U/mL)/streptomycin (100 mg/mL) and IL-2 (500U/ml). 0.5-1.0×10⁶ cells/test were collected by centrifugation (1500 rpm×5 minutes) and the supernatant was aspirated. The cells (single cell suspension) were washed with 4 mL ice cold FACS Buffer (PBS, 0.5-1% BSA, 0.1% NaN₃ sodium azide). The cells were re-suspended in 100 μL ice cold FACS Buffer and 5 μL antibody was added to each tube and incubated for 30 minutes on ice. The cells were washed 3 times by centrifugation at 1500 rpm for 5 minutes. The cells were then re-suspended in 500 μL ice cold FACS Buffer and temporarily kept in the dark on ice.

The cells were subsequently analyzed by flow cytometer (BD FACS Canto II) and the generated data were processed using FlowJo 7.6.2 software.

FACS analysis showed weak baseline expression of TRAIL on the NK cell surface.

TRAIL variant (D269H/195R) mRNA was synthesized by TriLink BioTechnologies, aliquoted and stored as −80° C. Mouse anti-human CD253-APC (Biolegend catalog number: 308210) and isotype control (Biolegend catalog number: 400122), antibodies were used to stain cell samples and were analyzed on a BD FACS Canto II flow cytometer. Propidium Iodide was used for cell viability. In order to achieve transfection efficiencies of up to 90% with high NK cell viability, an electroporation based technique was implemented using Maxcyte GT. Cells were processed in Maxcyte buffer prior to electroporation and cells were electroporated with 5-10 μg/ml of each individual mRNA (i.e. 5 μg/ml of CD19 CAR and 5 μg/ml TRAIL variant)

Naive or expanded NK cells were passaged one or two days before electroporation, as the cells must be in the logarithmic growth phase. 6-well plates were prepared by filling with 1.5 mL culture medium containing serum and supplements and pre-incubated in a humidified 37° C./5% CO₂ incubator. An aliquot of cell culture was prepared and the cells counted to determine the cell density. The required number of cells was centrifuged at 1500 rpm for 5 min, before discarding the supernatant completely. The cell pellet was re-suspended in room temperature Maxcyte buffer to a final concentration of 2×10⁶ cells/100p1 (maximum time in suspension=20 minutes). 100 μl cell suspension was mixed with 5 μg mRNA. The samples were transferred into Maxcyte-certified cuvettes OC-100×2 (making sure the samples covered the bottom of the cuvettes and avoiding air bubbles). The appropriate electroporation program was selected (i.e. NK-4). The cuvettes were then inserted into the cuvette holder. Immediately after the program had finished, in order to avoid damage to the cells, the cells were transferred to 6-well plates and incubated for 20 mins at 37° C. Flow cytometric analysis and cytotoxicity assays were performed 20-24 hours after electroporation. Flow cytometry staining was carried out as above.

Expression of CD19 CAR and TRAIL variant was shown to increase post-transfection, confirming the successful knock-in of the CD19 CAR and TRAIL variant genes into primary NK cells.

There was evidence of NK cell viability before and after transfection via electroporation. It can be seen that no statistically significant differences in cell viability are observed following transfection of the cells with TRAIL variant, confirming that the expression of variant TRAIL is not toxic to the cells.

The effects of the TRAIL variant on NK cell cytotoxicity were also measured. Mouse anti-human CD2-APC antibody (BD Pharmingen catalog number: 560642) was used. Annexin V-FITC antibody (ImmunoTools catalog number: 31490013) was used. A 96-well cell culture plate (SARSTEDT AG catalog number: 83.3922) was used. B-cell lymphoma cell lines OCI-LY10, RIVA, and SU-DHL6 were cultured in RPMI 1640 medium containing 10% FBS, 2 mM L-glutamine and penicillin (100 U/mL)/streptomycin (100 mg/mL).

The target cells were washed and pelleted via centrifugation at 1500 rpm for 5 minutes. Transfected NK cells were diluted to achieve a concentration of 2×10⁶/mL cells. The target cell density was then adjusted in pre-warmed RPMI 1640 medium, in order to produce effectortarget (E:T) ratios of 5:1, 2.5, and 1.25:1.

0.1 mL electroporated NK cells and 0.1 mL target cells were then mixed in a 96-well culture plate and placed in a humidified 37° C./5% CO₂ incubator for 16 hours. Flow cytometric analysis was then used to assay NK cell cytotoxicity; co-cultured cells (at different time points) were washed and then stained with CD2-APC antibody (5 μL/test), and cell viability was assessed using propidium iodide.

Data were further analyzed using FlowJo 7.6.2 software. CD2-positive and CD2-negative gates were set, which represented NK cell and target cell populations, respectively. The PI cells in the CD2-negative population were then analyzed for TRAIL-induced apoptosis.

The effects of NK cells expressing TRAIL variant on apoptosis were assessed for the three target cell lines: OCI-LY10, RIVA, and SU-DHL6, respectively. It is apparent for all target cell populations that TRAIL variant expression on NK cells increased the level of apoptosis, when compared to normal NK cells (not transfected with TRAIL variant).

Cells of the invention, expressing both the CD19 CAR and the TRAIL variant, offer a significant advantage in cancer therapy, due to their ability to specifically target cancer cells with high affinity and then kill those cells via the death receptor DR5. When challenged by the cells of the invention, cancer cells are prevented from developing defensive strategies to circumvent death via a single pathway. For example, cancers cannot effectively circumvent TRAIL-induced cell death by upregulating TRAIL decoy receptors, as cells of the invention are modified so that they remain cytotoxic in those circumstances.

### Example 7—Knockout of NK Cell TRAIL Receptors DR4 and DR5

NK cells are prepared as follows, having death receptor 5 (DR5) and/or death receptor 4 (DR4) function removed.

gRNA constructs targeting TRAIL-R2 (DR5) and TRAIL-R1 (DR4) are designed (e.g.
SEQ ID NO:1: CCCAUCUUGAACAUACCAG (DR5),
SEQ ID NO:2: AACCGGUGCACAGAGGGUGU (DR4) and
SEQ ID NO:3: AUUUACAAGCUGUACAUGGG (DR4))
   and prepared to target endogenous genes encoding DR5 and DR4 gene(s) in NK cells. CRISPR/Cas9 genome editing is then used to knock out the DR5 and/or DR4 target genes.

A total of 3 gRNA candidates are selected for the DR5 gene and their cleavage efficacies in RPMI8226 cells determined. A total of 3 gRNA candidates are selected for the DR4 gene and their cleavage efficacies in HL60 cells determined. RPMI8226 cells and HL60 are electroporated with the gRNA:Cas9 ribonucleoprotein (RNP) complex using Maxcyte® GT and subsequently knockout of DR5 and/or is analyzed by flowcytometry. The cleavage activity of the gRNA is also determined using an in vitro mismatch detection assay. T7E1 endonuclease recognizes and cleaves non-perfectly matched DNA, allowing the parental DR5 gene/DR4 gene to be compared to the mutated gene following CRISPR/Cas9 transfection and non-homologous end joining (NHEJ).

The gRNA with highest KO efficiency is selected to further experiments to knockout DR5/DR4 in primary NK cells, NK cell lines or CD34+ progenitors (for subsequent differentiation and expansion to NK cells). Knockout of DR4/DR5 is determined by flowcytometry based assays.

### Example 8—Knockdown of TRAIL Receptors DR4 and DR5 in NK Cells

siRNA knockdown of DR4 and/or DR5 in NK-92 cells, KHYG-1 cells and primary NK cells was performed by electroporation. siRNA based delivery was performed using the Maxcyte GT system.

The cells were then incubated in a humidified 37° C./5% CO₂ incubator until DR4 and/or DR5 knockdown analysis was performed. Flow cytometry analysis was performed 72 hours after electroporation, and (optionally) just prior to electroporation of TRAIL variant (e.g. E195R/D269H) mRNA in order to measure DR4 and/or DR5 expression levels. This electroporation protocol was found to reliably result in DR4 and DR5 knockdown in KHYG-1 cells, NK-92 cells and primary NK cells.

### Example 9—NK Cell Fratricide Resistance

As illustrated in FIG. 15, NK cell fratricide in the following cells was assessed: (1) primary NK cells, otherwise referred to as mock or wildtype NK cells, (2) primary NK cells expressing high affinity membrane-bound TRAIL ligand DR5^{E195R;D269H,} (3) primary NK cells with a DR5^{KD} via siRNA and (4) primary NK cells with a DR5^{KD} via siRNA and also expressing high affinity membrane-bound TRAIL ligand DR5^{E195R;D269H}.

Primary NK cells receiving the DR5 knockdown were electroporated with the DR5 siRNA on day 9 of the expansion, whereas primary NK cells receiving the DR5 TRAIL variant were electroporated with the variant mRNA on day 12 of the expansion.

It was observed that after prolonged expansion of the primary NK cells in IL-2 containing growth media that DR5 expression became upregulated, leading to increased fratricide when the DR5 TRAIL variant was expressed.

The data clearly indicate that knocking down DR5 expression using siRNA protects primary expanded NK cells from fratricide, regardless of whether those NK cells express wildtype TRAIL or the high-affinity DR5 TRAIL variant.

The invention thus provides highly cytotoxic NK cells for use in therapy.

1-33. (canceled)

34. A method of treating cancer comprising administering to a patient a natural killer (NK) cell or NK cell line that has been modified to have reduced function of Src homology 2 domain-containing protein tyrosine phosphatase 1 (SHP-1).

35. The method according to claim 34, wherein the NK cell or NK cell line modification is a genetic modification.

36. The method according to claim 34, wherein the NK cell or NK cell line modification involves genetically knocking down SHP-1 expression.

37. The method according to claim 34, wherein the NK cell or NK cell line modification is a transient modification.

38. The method according to claim 37, wherein the transient modification is an RNAi knockdown of SHP-1 expression.

39. The method according to claim 37, wherein the transient modification involves expressing an inactive form of SHP-1.

40. The method according to claim 39, wherein the inactive form of SHP-1 is expressed from an extra-chromosomal nucleic acid.

41. The method according to claim 39, wherein the inactive form of SHP-1 is expressed via an mRNA-based transfection system.

42. The method according to claim 41, wherein the mRNA-based transfection system is the Maxcyte GT system.

43. The method according to claim 34, wherein the NK cell or NK cell line modification involves expressing a dominant negative form of SHP-1.

44. The method according to claim 43, wherein the dominant negative form of SHP-1 is inducible.

45. The method according to claim 44, wherein expression of the dominant negative form of SHP-1 is induced by an antibody or doxycycline.

46. The method according to claim 34, wherein SHP-1 function is reduced by a phosphatase inhibitor.

47. The method according to claim 46, wherein the phosphatase inhibitor is a reversible inhibitor.

48. The method according to claim 46, wherein the phosphatase inhibitor is selected from tyrosine phosphatase inhibitor 1 (TPI-1), sodium stibogluconate (SSG), sodium orthovanadate (SOV), and sodium fluoride (NaF).

49. The method according to claim 34, wherein the NK cell is selected from an iPSC-derived NK cell and an umbilical cord-derived NK cell.

50. The method according to claim 34, wherein at least some of the cancer resides in a hypoxic environment.

51. The method according to claim 34, wherein the cancer is a blood cancer or a solid cancer.

52. The method according to claim 51, wherein the blood cancer is selected from acute lymphocytic leukemia (ALL), acute myeloid leukemia (AML), chronic lymphocytic leukemia (CLL), chronic myeloid leukemia (CIVIL), Hodgkin's lymphoma, non-Hodgkin's lymphoma, T-cell lymphoma, B-cell lymphoma, asymptomatic myeloma, multiple myeloma, active myeloma, and light chain myeloma.

53. A method of treating a solid cancer at least partially residing in a hypoxic environment, the method comprising administering to a patient a NK cell or NK cell line that has been modified to have reduced function of Src homology 2 domain-containing protein tyrosine phosphatase 1 (SHP-1).
